(19) United States
(12) Patent Application Publication
(10) Pub. No.: US 2024/0035070 A1
(43) Pub. Date: Feb. 1, 2024

(54) METHODS AND COMPOSITIONS FOR IN SITU ANALYSIS USING TIME-GATED DETECTION

(71) Applicant: 10x Genomics, Inc., Pleasanton, CA (US)

(72) Inventor: Cheyenne CHRISTOPHERSON, San Ramon, CA (US)

(21) Appl. No.: 18/360,135

(22) Filed: Jul. 27, 2023

Related U.S. Application Data
(60) Provisional application No. 63/393,040, filed on Jul. 28, 2022.

(51) Int. Cl. C12Q 1/6837; C12Q 1/6841

(52) U.S. Cl. CPC C12Q 1/6837; C12Q 1/6841

(57) ABSTRACT

The present disclosure generally relates to methods and compositions for detecting a plurality of molecules of one or more analytes in a sample. In some aspects, the present disclosure relates to methods for determining the locations and identities of analytes in a biological sample using detectably labeled probes comprising detectable labels with different signal emission lifetimes. In some aspects, the present disclosure relates to methods for identifying the detectable labels of the detectably labeled probes using time-gated detection. The methods herein have particular applicability in the detection of identifier sequences (e.g., analyte sequences or barcode sequences) in situ in a biological sample, including those using sequential cycles of detectably labeled probe hybridization to decode the identifier sequences.

### CROSS-REFERENCE TO RELATED APPLICATIONS

This application claims priority to U.S. Provisional Patent Application No. 63/393,040, filed Jul. 28, 2022, entitled “METHODS AND COMPOSITIONS FOR IN SITU ANALYSIS USING TIME-GATED DETECTION,” which is herein incorporated by reference in its entirety for all purposes.

### FIELD

The present disclosure generally relates to methods and compositions for detecting a plurality of molecules of one or more analytes at multiple locations in a sample.

### BACKGROUND

Genomic, transcriptomic, and proteomic profiling of cells and tissue samples using microscopic imaging can detect multiple analytes of interest at the same time, thereby providing valuable information regarding analyte abundance and localization in situ. Thus, in situ assays are important tools, for example, for understanding the molecular basis of cell identity and developing treatment for diseases. There is a need for new and improved methods for in situ assays. Provided herein are methods and compositions that address such and other needs.

### SUMMARY

Plex-scalability of in situ detection methods can be limited by the number of detection channels. For example, in situ detection with multi-cycle decoding may rely on detection of different detectable labels in each cycle, typically using 4 detectable labels, each detected in a different detection channel, for example for detecting emissions of different spectra (colors), e.g., at 488, 532, 590, or 647 nm. Typically after n number of cycles using d number of distinguishable detectable labels, the number of different analytes that can be decoded is d\hat{ }n (e.g., 4\hat{ }n in the case of four different fluorophores each of a different color). Thus, the analyte encoding capacity can be limited by the number of different detectable labels that are available. In order to increase the encoding capacity, one approach is to increase the number of probe hybridization cycles n. However, between cycles, detectably labeled probes typically need to be stripped from the sample or their signals extinguished before new ones are added for hybridization and detection. Repeated cycles of detectably labeled probe hybridization (often with probe stripping) can dramatically increase the overall time it takes to detect a large number of different genes. There is a need for methods that reduce the number of probe hybridization cycles and the overall decoding time required to decode a large number of analytes. Provided herein include compositions and methods multi-cycle decoding with time-gated detection, where the methods comprise the detection of detectable labels using different time gates (e.g., based on the different signal emission lifetimes of the detectable labels) and generating signal code sequences with signal codes corresponding to the detectable labels detected via time-gated detection. In some aspects, by using a combination of different emissions spectra and different signal emission lifetimes of the detectable labels, time-gated detection allows the use of more “colors” (detectable labels that are distinguishable from one another) per cycle compared to typical 4-color channel fluorescent microscopy. Since the number of distinguishable detectable labels d can be increased using time-gated detection, the number of probe hybridization cycles n can be decreased and still achieve the same or even higher number of different analytes that can be decoded, thereby decreasing the overall decoding time.

In some embodiments, provided herein is a method for analyzing a biological sample, comprising: a) contacting the biological sample with a plurality of detectably labeled probes for detecting multiple analytes, wherein each detectably labeled probe is configured to directly or indirectly bind to a different analyte or product thereof and comprises a detectable label, and wherein the detectable labels of the plurality of detectably labeled probes comprise a first detectable label having a first signal emission lifetime and a second detectable label having a second signal emission lifetime that is longer than the first signal emission lifetime; b) detecting signals associated with the detectable labels, or absence thereof, during a detection time interval t1 and a detection time interval t2 at one or more locations in the biological sample, wherein the onset of t2 is later than the onset of t1, wherein signals associated with the first detectable label are detectable during t1 and not during t2, and wherein signals associated with the second detectable label are detectable during t2; and c) generating a signal code sequence comprising signal codes corresponding to the signals or absence thereof detected in step b) during t1 and t2, respectively, at the one or more locations, wherein the signal code sequence corresponds to an analyte of the multiple analytes, thereby identifying the analyte at the one or more locations in the biological sample.

In any of the embodiments herein, the detectable labels of the plurality of detectably labeled probes can comprise a third detectable label having a third signal emission lifetime that is longer than the second signal emission lifetime. In any of the embodiments herein, a detecting in step can further comprise: detecting signals associated with the detectable labels, or absence thereof, during a detection time interval t3 at one or more locations in the biological sample, wherein the onset of t3 is later than the onset of t2, wherein signals associated with the first detectable label and second detectable label are not detectable during t3, and wherein signals associated with the third detectable label are detectable during t3. In any of the embodiments herein, the signal codes may further correspond to the signals or absence thereof that are detected during time interval t3 at the one or more locations.

In some embodiments, provided herein is a method for analyzing a biological sample, comprising: a) contacting the biological sample with a plurality of first detectably labeled probes for detecting multiple analytes, wherein each first detectably labeled probe is configured to directly or indirectly bind to a different analyte or product thereof and comprises a detectable label; b) detecting signals associated with the detectable labels of the plurality of first detectably labeled probes, or absence of the signals, during a detection time interval t1 and a detection time interval t2, at one or more locations in the biological sample, wherein the detectable labels of the plurality of first detectably labeled probes comprise a first detectable label having a first signal emission lifetime and a second detectable label having a second signal emission lifetime that is longer than the first signal emission lifetime, wherein the onset of t2 is later than the onset of t1, wherein signals associated with the first detectable label are detectable during t1 and not t2, and wherein signals associated with the second detectable label are detectable during t2; c) contacting the biological sample with a plurality of subsequent detectably labeled probes for detecting the multiple analytes, wherein each subsequent detectably labeled probe is configured to directly or indirectly bind to a different analyte or product thereof and comprises a detectable label; and d) detecting signals associated with the detectable labels of the plurality of subsequent detectably labeled probes, or absence of the signals, during a subsequent detection time interval t1s and a subsequent detection time interval t2s, at the one or more locations in the biological sample, wherein the detectable labels of the plurality of subsequent detectably labeled probes comprise a subsequent first detectable label having a subsequent first signal emission lifetime and a subsequent second detectable label having a subsequent second signal emission lifetime that is longer than the subsequent first signal emission lifetime, wherein the onset of t2s is later than the onset of t1s, wherein signals associated with the subsequent first detectable label are detectable during t1s and not t2s, and wherein signals associated with the subsequent second detectable label are detectable during t2s; wherein a signal code sequence is generated that comprises signal codes corresponding to the signals or absence thereof detected in step b) during t1 and t2, respectively, and step d) during t1s and t2s, respectively, at the one or more locations, wherein the signal code sequence corresponds to an analyte of the multiple analytes, thereby identifying the analyte at the one or more locations in the biological sample.

In any of the embodiments herein, the analyte can be a first analyte, the signal code sequence can be a first signal code sequence, and a second signal code sequence can be generated for a second analyte, the second signal code sequence corresponding to the signals or absence thereof detected at the one or more locations.

In any of the embodiments herein, the detection time intervals t1 and t2 may comprise the same two time intervals as the subsequent detection time intervals t1s and t2s, respectively. In any of the embodiments herein, the detection time intervals t1 and t2 do not need to comprise the same two time intervals as the subsequent detection time intervals t1s and t2s, respectively.

In any of the embodiments herein, the first and second detectable labels can comprise the same two detectable labels as the subsequent first and subsequent second detectable labels. In any of the embodiments herein, the first and second detectable labels can comprise at least one detectable label that is not comprised by the subsequent first and subsequent second detectable labels. In any of the embodiments herein, each detectable label can emit a detectable signal in response to a stimulus. In any of the embodiments herein, the stimulus for each detectable label can be electromagnetic radiation, optionally light. In any of the embodiments herein, the stimulus for any two or more detectable labels can be the same or different. In any of the embodiments herein, any two or more detectable labels can be stimulated simultaneously or non-simultaneously. In any of the embodiments herein, the detectable signal emitted by each detectable label can be electromagnetic radiation, optionally light, optionally photoluminescence, optionally fluorescence and/or phosphorescence. In any of the embodiments herein, any two or more of the detectable labels can emit light of the same wavelength. In any of the embodiments herein, any two or more of the detectable labels can emit light of different wavelengths.

In any of the embodiments herein, the signal emission lifetime for each detectable label can be the length of time between a) the offset of the stimulus for the detectable label, and b) the time at which the detectable label no longer emits a detectable signal in response to the stimulus. In any of the embodiments herein, the signal emission lifetime of each detectable label can be independently selected from: less than about 10 μs, between about 10 μs and about 100 μs, between about 100 μs and about 300 μs, between about 300 μs and about 1 ms, and greater than about 1 ms. In any of the embodiments herein, the onset and offset of each detection time interval can be defined with respect to the offset of the stimulus for the detectable label being detected.

In any of the embodiments herein, the signals associated with the detectable labels can be detected using one or more detection channels, each detection channel being configured to detect light from a different range of wavelengths. In any of the embodiments herein, any two or more of the signals associated with the detectable labels can be detected using the same detection channel or different detection channels.

In any of the embodiments herein, one or more of the detectable labels can comprise a fluorophore. In any of the embodiments herein, one or more of the detectable labels can comprise a phosphor. In any of the embodiments herein, one or more of the detectable labels can comprise a thermally activated delayed fluorescence (TADF) emitter. In any of the embodiments herein, one or more of the detectable labels can comprise a phosphorescent emitter. In any of the embodiments herein, one or more of the detectable labels can comprise a thermally activated delayed fluorescence (TADF) emitter and/or a phosphorescent emitter. In any of the embodiments herein, each emitter (e.g., a TADF or phosphorescent emitter) comprised by a detectable label can independently comprise one or more of any of the moieties selected from: DABNA-1; DABNA-2; DTC-DPS; DMAC-DPS; 4CzIPN; NAI-DMAC; DPA-DCPP; an organoboron moiety; a cyanobenzene moiety; a dicyanobenzene moiety; a diphenyltriazine moiety; a diphenylsulfone moiety; a naphthalimide moiety; a dicyanopyrazino moiety; a phenanthrene moiety; a carbazole moiety optionally substituted with C₁-C₆ alkyl; a phenoxazine moiety optionally substituted with C₁-C₆ alkyl; a triphenylamine moiety optionally substituted with C₁-C₆ alkyl; a diphenylamide moiety optionally substituted with C₁-C₆ alkyl; an acridine moiety optionally substituted with C₁-C₆ alkyl; and a dimethylacridine moiety optionally substituted with C₁-C₆ alkyl. In any of the embodiments herein, one or more of the emitters (e.g., a TADF or phosphorescent emitter) comprised by a detectable label can be encapsulated in a polymer matrix, optionally forming a polymer dot and/or an organic dot. In any of the embodiments herein, the polymer matrix can comprise a semiconducting polymer. In any of the embodiments herein, the semiconducting polymer can harvest energy from photons and transfer energy to the emitter. In any of the embodiments herein, the emitter (e.g., a TADF or phosphorescent emitter) encapsulated in a semiconducting polymer, in comparison to the same emitter not encapsulated in a semiconducting polymer, can exhibit reduced variance in emission lifetime, reduced oxygen quenching, increased brightness, increased photostability, increased molar absorptivity, and/or increased quantum yield. In any of the embodiments herein, the polymer matrix can comprise a water-soluble polymer. In any of the embodiments herein, the polymer matrix can allow for specific biological targeting. In some of any such embodiments, each detectably labeled probe comprises at least two different detectable labels. In some instances, a detectably labeled probe is associated with a polymer dot comprising at least two different detectable labels.

In any of the embodiments herein, each analyte can be independently a nucleic acid analyte or non-nucleic acid analyte. In any of the embodiments herein, each detectably labeled probe can (a) bind to a primary probe that directly binds to its corresponding analyte, or (b) bind to an intermediate probe that binds directly or indirectly to a primary probe that directly binds to its corresponding analyte. In any of the embodiments herein, each primary probe and each intermediate probe can be independently selected from the group consisting of: a probe comprising a 3′ or 5′ overhang, optionally wherein the 3′ or 5′ overhang comprises one or more barcode sequences; a probe comprising a 3′ overhang and a 5′ overhang, optionally wherein the 3′ overhang and the 5′ overhang each independently comprises one or more barcode sequences; a circular probe; a circularizable probe or probe set; a probe or probe set comprising a split hybridization region configured to hybridize to a splint, optionally wherein the split hybridization region comprises one or more barcode sequences; and a combination thereof. In any of the embodiments herein, the product of each analyte can be a rolling circle amplification (RCA) product generated in situ in the biological sample.

In any of the embodiments herein, the biological sample can be non-homogenized and optionally selected from the group consisting of a fixed tissue sample, a formalin-fixed, paraffin-embedded (FFPE) sample, a frozen tissue sample, and a fresh tissue sample. In any of the embodiments herein, the biological sample can be a fixed biological sample. In any of the embodiments herein, the biological sample is not fixed. In any of the embodiments herein, the biological sample can be permeabilized. In any of the embodiments herein, the biological sample can be embedded in a matrix, optionally wherein the matrix comprises a hydrogel. In any of the embodiments herein, the biological sample can be cleared, optionally wherein the clearing comprises contacting the biological sample with a proteinase. In any of the embodiments herein, the biological sample can be crosslinked. In any of the embodiments herein, the biological sample can be a tissue slice between about 1 μm and about 50 μm in thickness, optionally wherein the tissue slice is between about 5 μm and about 35 μm in thickness. In any of the embodiments herein, the method can be performed in situ in the biological sample. In any of the embodiments herein, one or more of the signals can be detected in situ in the biological sample.

In some embodiments, provided herein is a kit comprising a plurality of detectably labeled probes for detecting multiple analytes, wherein each detectably labeled probe is configured to directly or indirectly bind to a different analyte or product thereof and comprises a detectable label, and wherein the detectable labels of the plurality of detectably labeled probes comprise a first detectable label having a first signal emission lifetime and a second detectable label having a second signal emission lifetime that is longer than the first signal emission lifetime. In any of the embodiments herein, the kit can comprise instructions to detect signals associated with the detectable labels, or absence thereof, during a detection time interval t1 and a detection time interval t2, wherein the onset of t2 is later than the onset of t1, wherein signals associated with the first detectable label are detectable during t1 and not during t2, and wherein signals associated with the second detectable label are detectable during t2. In any of the embodiments herein, one or more of the detectable labels can comprise a fluorophore. In any of the embodiments herein, one or more of the detectable labels can comprise a thermally activated delayed fluorescence (TADF) emitter. In any of the embodiments herein, one or more of the detectable labels can comprise a fluorophore and/or phosphor. In any of the embodiments herein, one or more of the detectable labels can comprise a thermally activated delayed fluorescence (TADF) emitter and/or a phosphorescent emitter.

### BRIEF DESCRIPTION OF THE DRAWINGS

The drawings illustrate certain features and advantages of this disclosure. These embodiments are not intended to limit the scope of the appended claims in any manner.

FIGS. 1A-1C show schematics illustrating an example of time-gated detection of detectable labels with different signal emission lifetimes.

FIGS. 2A-2D show examples of detection time intervals that can be used for time-gated detection and decoding of detectable labels. FIG. 2E provides a summary: the first label is detected during time interval t1 and not time interval t2, whereas the second label is detected during time intervals t1 and t2.

FIG. 3 shows a schematic illustrating “traditional” multi-cycle decoding of analytes using four detectable labels each with a different signal emission spectrum.

FIG. 4A shows a schematic illustrating an example of cycles of multi-cycle decoding of analytes using twelve detectable labels collectively having four different emission spectra and three different signal emission lifetimes (T). FIG. 4B shows a schematic illustrating an example for decoding analytes with multi-color detectably labeled probes (e.g., using six detectable labels enabled by combining three detection channels with two different signal emission time bins).

FIG. 5 shows a schematic illustrating exemplary photophysical pathways for a thermally activated delayed fluorescent (TADF) emitter.

FIG. 6 shows chemical structures of exemplary TADF emitters.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Plex-scalability of in situ detection methods can be limited by the number of detection channels. For example, in situ sequencing with multi-cycle decoding relies on detection of different detectable labels in each cycle, typically using 4 detectable labels, each detected in a different detection channel, e.g. for detecting emissions of different spectra. After n number of cycles using d number of detectable labels, the number of different analytes that can be decoded is d\hat{ }n. Between cycles, detectably labeled probes are stripped from the sample before new ones are added for hybridization and detection. Repeated cycles of stripping and hybridization dramatically increases the overall time it takes to detect a large number of different genes.

Increasing the number of detectable labels that can be detected per cycle would greatly increase the number of genes that can be detected without increasing the number of cycles. For example, in an assay with d=4 detectable labels and n=3 cycles, theoretically the maximum number of analytes that can be decoded is d\hat{ }n, or 4\hat{ }3=64 analytes. In contrast, in an assay with (1=5 detectable labels and the same number of cycles, the maximum number of analytes that can be decoded can be 5\hat{ }3=125 analytes.

The number of detectable labels that can be detected per cycle may be increased by using detectable labels with different emission lifetimes, detected using time-gated imaging and decoding. For example, two different detectable labels can be used that are detectable in the same detection channel (e.g., both emit fluorescent signals detectable in a 488 nm channel) and emit fluorescent signal for different amounts of time after excitation (e.g., the detectable labels have different emission lifetimes). Determining the presence or absence of a detectable signal in the 488 nm channel during different detection time intervals (e.g., time bins) after excitation would allow for the detection of the different detectable labels using a single detection channel, based on the detection of discrete time-gated signals that arrive at a detector of a microscope at different times. For example, using a first detectable label that emits for 50 μs, and a second detectable label that emits for 100 μs, the first detectable label would be detected in a 30-50 μs detection time interval but not detected in a 80-100 μs interval. In contrast, the second detectable label would be detected in both the 30-50 μs detection time interval and the 80-100 μs detection time interval. Thus, using detectable labels with different emission lifetimes in multi-cycle decoding with time-gated detection allows for a larger number of detectable labels to be used per cycle, without necessarily increasing the number of detection channels.

Time-gated detection can also be performed in each different detection channel (e.g., color). In some embodiments, time-gated detection can distinguish detectable labels having partially or completely overlapping emission spectra, provided that the detectable labels have different signal emission lifetimes that are distinguishable using detection within discrete time bins. For example, using the standard 4 channels, with 2 detectable labels differentiated by time-gated detection in each channel, 8 different detectable labels can be detected per cycle, allowing for a maximum of 8\hat{ }3=512 analytes to be decoded in a 3-cycle assay. In comparison, in a standard 3-cycle assay using 4 detectable labels without multi-cycle decoding with time-gated detection, the maximum number of decoded analytes is 64. The experiment can be done with any number of time detection windows and detectable labels. In some aspects, any suitable number of time-gated channels (e.g., number of detectable labels having different emission spectra and/or different signal emission lifetimes) can be used, and the number of time-gated channels can be selected based on the instrument being used. In some aspects, significantly more genes can be detected per cycle with the addition of multi-cycle decoding with time-gated detection, in comparison to traditional multi-cycle decoding, limiting the number of stripping and hybridization steps required to decode a large number of genes, and reducing the overall time needed to perform the experiment.

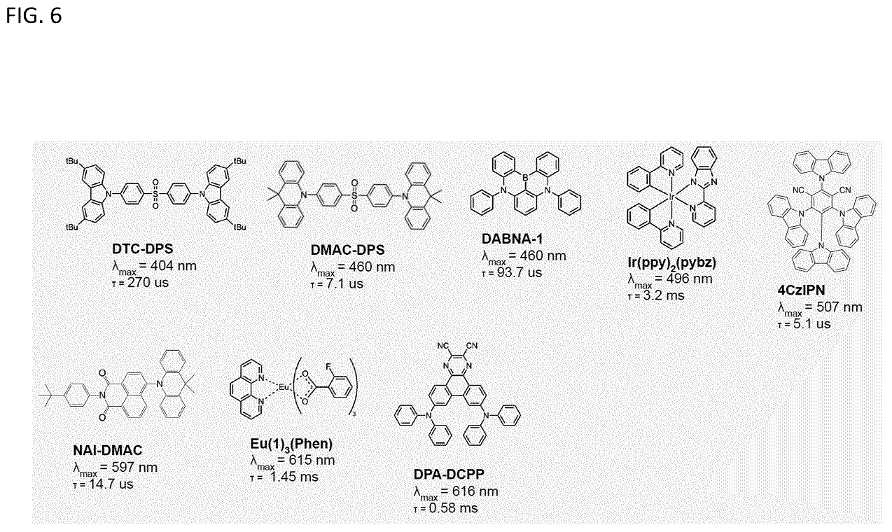

Detectable labels with different emission spectra and lifetimes may comprise any suitable emitter. Emitters can include fluorophores that emit wavelengths of at or about 488 nm, 532 nm, 590 nm, or 647 nm. Emitters can also include thermally activated delayed fluorescence (TADF) emitters and/or organometallic phosphorescent emitters. Emitters can be introduced as a colloidal nanoprecipitation suspension in water. Emitters can be incorporated into nanoparticles (e.g. as functionalized polymer dots or organic dots). Emitters may comprise any suitable emitter, including those shown in FIG. 6, including: DTC-DPS; DMAC-DPS; DABNA-1; Ir(ppy)₂(pybz); 4CzIPN; NAI-DMAC; Eu(1)₃(Phen); or DPA-DCPP.

Different detectable labels, for example detectable labels comprising any of the emitters described herein, can theoretically be distinguished based on analysis of emission signatures, whereby the intensities and wavelengths of emissions are traced over time, delineating an emission curve having various properties and kinetics. In contrast, using multi-cycle decoding with time-gated detection as described herein, signals are either detected or not detected within a discrete detection time interval. In some embodiments, a method disclosed herein comprises using discrete detection time intervals (e.g., time bins), and detecting within those time intervals. In some embodiments, multi-cycle decoding with time-gated detection as described herein comprises generating a signal code sequence comprising signal codes corresponding to the signals or absence thereof, wherein the detection of each discrete signal code in the signal code sequence does not rely on a lifetime measurement of an intensity-based and time-based signature of any particular detectable label. In some embodiments, multi-cycle decoding with time-gated detection as described herein does not rely on the detection of a continuous lifetime emission curve in order to distinguish the various detectable labels in the same probe hybridization and detection cycle. For example, time-gated detection provided herein limits exposure time and uses certain bins of time for capturing images rather than a longer exposure and continuous capture needed for detection of a continuous lifetime emission curve. In some aspects, multi-cycle decoding with time-gated detection requires less processing, analysis and computational power, while still allowing for different detectable labels to be distinguished within a detection channel, e.g., without the need to analyze photon arrival timelines in order to distinguish detectable labels having overlapping emission spectra. In some aspects, multi-cycle decoding with time-gated detection requires fewer images to be acquired, stored, and processed. This provides practical opportunities for further increasing the plexity of in situ assays.

In some embodiments, provided herein is a method for analyzing a biological sample. In some embodiments, the method comprises contacting the biological sample with a plurality of detectably labeled probes for detecting multiple analytes. In some embodiments, each detectably labeled probe is configured to directly or indirectly bind to a different analyte or product thereof and comprises a detectable label. In some embodiments, the detectable labels of the plurality of detectably labeled probes comprise a first detectable label having a first signal emission lifetime and a second detectable label having a second signal emission lifetime that is longer than the first signal emission lifetime. In some embodiments, the method comprises detecting signals associated with the detectable labels, or absence thereof, during a detection time interval t1 and a detection time interval t2 at one or more locations in the biological sample. In some embodiments, the onset of t2 is later than the onset of t1. In some embodiments, signals associated with the first detectable label are detectable during t1 and not t2. In some embodiments, signals associated with the second detectable label are detectable during detection time interval t2. In some embodiments, the method comprises generating a signal code sequence comprising signal codes corresponding to the signals or absence thereof detected during t1 and t2, respectively, at the one or more locations. In some embodiments, the signal code sequence corresponds to an analyte of the multiple analytes, thereby identifying the analyte at the one or more locations in the biological sample.

In some embodiments, the detectable labels of the plurality of detectably labeled probes comprise a third detectable label having a third signal emission lifetime that is longer than the second signal emission lifetime. In some embodiments, the detecting further comprises detecting signals associated with the detectable labels, or absence thereof, during a detection time interval t3 at one or more locations in the biological sample. In some embodiments, the onset of t3 is later than the onset of t2. In some embodiments, signals associated with the first detectable label and second detectable label are not detectable during t3. In some embodiments, signals associated with the third detectable label are detectable during t3. In some embodiments, the signal codes further correspond to the signals or absence thereof detected during t3 at the one or more locations.

In some embodiments, provided herein is a method for analyzing a biological sample. In some embodiments, the method comprises contacting the biological sample with a plurality of first detectably labeled probes for detecting multiple analytes. In some embodiments, each first detectably labeled probe is configured to directly or indirectly bind to a different analyte or product thereof and comprises a detectable label. In some embodiments, the method comprises detecting signals associated with the detectable labels of the plurality of first detectably labeled probes, or absence of the signals, during a detection time interval t1 and a detection time interval t2, at one or more locations in the biological sample. In some embodiments, the detectable labels of the plurality of first detectably labeled probes comprise a first detectable label having a first signal emission lifetime and a second detectable label having a second signal emission lifetime that is longer than the first signal emission lifetime. In some embodiments, the onset of t2 is later than the onset of t1. In some embodiments, signals associated with the first detectable label are detectable during t1 and not t2, and signals associated with the second detectable label are detectable during t2. In some embodiments, the method further comprises contacting the biological sample with a plurality of subsequent detectably labeled probes for detecting the multiple analytes, wherein each subsequent detectably labeled probe is configured to directly or indirectly bind to a different analyte or product thereof and comprises a detectable label. In some embodiments, the method further comprises detecting signals associated with the detectable labels of the plurality of subsequent detectably labeled probes, or absence of the signals, during a subsequent detection time interval t1s and a subsequent detection time interval t2s, at the one or more locations in the biological sample. In some embodiments, the detectable labels of the plurality of subsequent detectably labeled probes comprise a subsequent first detectable label having a subsequent first signal emission lifetime and a subsequent second detectable label having a subsequent second signal emission lifetime that is longer than the subsequent first signal emission lifetime. In some embodiments, the onset of t2s is later than the onset of t1s, signals associated with the subsequent first detectable label are detectable during t1s and not t2s, and signals associated with the subsequent second detectable label are detectable during t2s. In some embodiments, a signal code sequence is generated that comprises signal codes corresponding to the signals or absence thereof detected during t1 and t2, respectively, and during t1s and t2s, respectively, at the one or more locations. In some embodiments, the signal code sequence corresponds to an analyte of the multiple analytes, thereby identifying the analyte at the one or more locations in the biological sample.

In some embodiments, one of the plurality of first detectably labeled probes and one of the plurality of subsequent detectably labeled probes are configured to directly or indirectly bind to the same analyte or product thereof. In some embodiments, each first detectably labeled probe has a corresponding second detectably labeled probe, and the corresponding detectably labeled probes are configured to directly or indirectly bind to the same analyte or product thereof. In some embodiments, an analyte is targeted (e.g., directly or indirectly bound) by a first detectably labeled probe in a first cycle, and the same analyte is targeted (e.g., directly or indirectly bound) by a second detectably labeled probe in a second cycle. The first and second detectably labeled probes can have the same detectable label or different detectable labels. In some embodiments, the first and second detectably labeled probes can have detectable labels of the same or similar signal emission spectra (e.g., emitters of the same or similar λₘₐₓ and detected as the same or similar colors) and different signal emission lifetimes (e.g., τ of one emitter being at least about 2, at least about 4, at least about 6, at least about 8, or at least about 10 times of the other emitter). In some embodiments, the first and second detectably labeled probes can have detectable labels of different signal emission spectra (e.g., emitters detected as different colors) and the same or similar signal emission lifetimes (e.g., emitters of the same or similar τ and detected in the same time bin(s)). In some embodiments, the first and second detectably labeled probes can have detectable labels of different signal emission spectra (e.g., emitters detected as different colors) and different signal emission lifetimes (e.g., τ of one emitter being at least about 2, at least about 4, at least about 6, at least about 8, or at least about 10 times of the other emitter).

In some embodiments, the analyte is a first analyte, the signal code sequence is a first signal code sequence, and wherein a second signal code sequence is generated for a second analyte, the second signal code sequence corresponding to the signals or absence thereof detected at the one or more locations.

In some embodiments, the detection time intervals t1 and t2 comprise the same two time intervals as the subsequent detection time intervals t1s and t2s, respectively. In some embodiments, the detection time intervals t1 and t2 do not comprise the same two time intervals as the subsequent detection time intervals t1s and t2s, respectively. In some embodiments, the first and second detectable labels comprise the same two detectable labels as the subsequent first and subsequent second detectable labels. In some embodiments, the first and second detectable labels comprise at least one detectable label that is not comprised by the subsequent first and subsequent second detectable labels. In some embodiments, each detectable label emits a detectable signal in response to a stimulus. In some embodiments, each detectable label is electromagnetic radiation, optionally light. In some embodiments, the stimulus for any two or more detectable labels is the same or different. In some embodiments, any two or more detectable labels are stimulated simultaneously or non-simultaneously. In some embodiments, the detectable signal emitted by each detectable label is electromagnetic radiation, optionally light, optionally photoluminescence (PL, light emission from any form of matter after the absorption of photons (electromagnetic radiation)), optionally fluorescence and/or phosphorescence. In some embodiments, the detectable signal emitted by one or more of the detectable labels used in time-gated detection disclosed herein is fluorescence. In some embodiments, the detectable signal emitted by one or more of the detectable labels used in time-gated detection disclosed herein is phosphorescence. In some embodiments, any two or more of the detectable labels emit light of the same wavelength. In some embodiments, any two or more of the detectable labels emit light of different wavelengths. In some embodiments, the signal emission lifetime for each detectable label is the length of time between a) the offset of the stimulus for the detectable label, and b) the time at which the detectable label no longer emits a detectable signal in response to the stimulus. In some embodiments, the signal emission lifetime of each detectable label is independently selected from: less than about 10 μs, between about 10 μs and about 100 μs, between about 100 μs and about 300 μs, between about 300 μs and about 1 ms, and greater than about 1 ms. In some embodiments, the onset and offset of each detection time interval is defined with respect to the offset of the stimulus for the detectable label being detected. In some embodiments, the signals associated with the detectable labels are detected using one or more detection channels, each detection channel being configured to detect light from a different range of wavelengths. In some embodiments, any two or more of the signals associated with the detectable labels are detected using the same detection channel or different detection channels.

In some embodiments, one or more of the detectable labels comprise a fluorophore. In some embodiments, one or more of the detectable labels comprise a phosphor. In some embodiments, one or more of the detectable labels comprise a thermally activated delayed fluorescence (TADF) emitter and/or a phosphorescent emitter. In some embodiments, each TADF emitter comprised by a detectable label independently comprises one or more of any of the moieties selected from: DABNA-1; DABNA-2; DTC-DPS; DMAC-DPS; 4CzIPN; NAI-DMAC; DPA-DCPP; an organoboron moiety; a cyanobenzene moiety; a dicyanobenzene moiety; a diphenyltriazine moiety; a diphenylsulfone moiety; a naphthalimide moiety; a dicyanopyrazino moiety; a phenanthrene moiety; a carbazole moiety optionally substituted with C₁-C₆ alkyl; a phenoxazine moiety optionally substituted with C₁-C₆ alkyl; a triphenylamine moiety optionally substituted with C₁-C₆ alkyl; a diphenylamide moiety optionally substituted with C₁-C₆ alkyl; an acridine moiety optionally substituted with C₁-C₆ alkyl; and a dimethylacridine moiety optionally substituted with C₁-C₆ alkyl.

In some embodiments, one or more of the TADF emitters and/or phosphorescent emitters comprised by a detectable label is encapsulated in a polymer matrix, optionally forming a polymer dot and/or an organic dot. In some embodiments, the polymer matrix comprises a semiconducting polymer. In some embodiments, the semiconducting polymer harvests energy from photons and transfers energy to the TADF emitter. In some embodiments, the TADF emitter encapsulated in a semiconducting polymer, in comparison to the same TADF emitter not encapsulated in a semiconducting polymer, exhibits reduced variance in emission lifetime, reduced oxygen quenching, increased brightness, increased photostability, increased molar absorptivity, and/or increased quantum yield. the polymer matrix comprises a water-soluble polymer. In some embodiments, the polymer matrix allows for specific biological targeting.

In some embodiments, the polymer dot comprises an amphiphilic polymer. In some embodiments, the polymer dot comprising an amphiphilic polymer comprises at or about 30%-40% by weight, at or about 40%-50% by weight, at or about 50%-60% by weight, at or about 60%-70% by weight, or more, of a semiconducting polymer. In some embodiments, the polymer dot comprising an amphiphilic polymer comprises at or about 50% by weight of a semiconducting polymer. In some embodiments, the semiconducting polymer serve as an energy capture source which then transfers energy to a lower-power laser (LPL) emitter (e.g., a TADF emitter or a phosphorescent emitter disclosed herein) to increase its brightness.

In some embodiments, each polymer dot and/or organic dot may comprise at least two different TADF emitters and/or phosphorescent emitters. In some aspects, multi-colored detectably labeled probes can encode higher information density than using a single color. In some cases, a polymer dot that compresses many emitters into one nanoparticle may be brighter and more photostable than TADF emitters not encapsulated in a polymer matrix. In some aspects, the use of spectrally uncoupled fluorophores may allow for multi-colored detection without energy transfer to the lowest energy emitter. In some embodiments, hydrophobic emitters are encapsulated into a polymer dot and/or an organic dot for water-compatibility. In some cases, the corona of the polymer dot or an organic dot is functionalized for specific binding (e.g., for directly or indirectly binding to an analyte or product thereof). In some aspects, the detectably labeled probe comprises or is associated with a polymer dot.

In some embodiments, each analyte is independently a nucleic acid analyte or non-nucleic acid analyte. Each probe is independently i) a primary probe that directly binds to its corresponding analyte, or ii) a probe that directly or indirectly binds to the primary probe. The primary probe and the probe that directly or indirectly binds to the primary probe are independently selected from the group consisting of: a probe comprising a 3′ or 5′ overhang, optionally wherein the 3′ or 5′ overhang comprises one or more barcode sequences; a probe comprising a 3′ overhang and a 5′ overhang, optionally wherein the 3′ overhang and the 5′ overhang each independently comprises one or more barcode sequences; a circular probe; a circularizable probe or probe set; a probe or probe set comprising a split hybridization region configured to hybridize to a splint, optionally wherein the split hybridization region comprises one or more barcode sequences; and a combination thereof. In some instances, the product of each analyte is a rolling circle amplification (RCA) product generated in situ in the biological sample.

In some embodiments, the biological sample is non-homogenized and optionally selected from the group consisting of a formalin-fixed, paraffin-embedded (FFPE) sample, a frozen tissue sample, and a fresh tissue sample. the biological sample is fixed or not fixed. the biological sample is permeabilized. In some embodiments, the biological sample is embedded in a matrix, optionally wherein the matrix comprises a hydrogel. In some embodiments, the biological sample is cleared, optionally wherein the clearing comprises contacting the biological sample with a proteinase. In some embodiments, the biological sample is crosslinked. In some embodiments, the biological sample is a tissue slice between about 1 μm and about 50 μm in thickness, optionally wherein the tissue slice is between about 5 μm and about 35 μm in thickness.

In some embodiments, the method is performed in situ in the biological sample. In some embodiments, one or more of the signals are detected in situ in the biological sample.

### II. Detectably Labeled Probes and Detectable Labels

In some aspects, provided herein are detectably labeled probes each comprising a detectable label. In some embodiments, provided herein are detectably labeled probes each configured to directly or indirectly bind to a different analyte or product thereof and comprises a detectable label. In some embodiments, a detectably labeled probe disclosed herein binds directly or indirectly to a target sequence. In some embodiments, a detectably labeled probe may bind a primary probe that directly binds to its corresponding analyte, or products generated using the primary probe. In some embodiments, a detectably labeled probe may bind a probe that directly or indirectly binds to the primary probe or products generated using the primary probe. For example, the primary probe and the probe that directly or indirectly binds to the primary probe are independently selected from the group consisting of: a probe comprising a 3′ or 5′ overhang, optionally wherein the 3′ or 5′ overhang comprises one or more barcode sequences; a probe comprising a 3′ overhang and a 5′ overhang, optionally wherein the 3′ overhang and the 5′ overhang each independently comprises one or more barcode sequences; a circular probe; a circularizable probe or probe set; a probe or probe set comprising a split hybridization region configured to hybridize to a splint, optionally wherein the split hybridization region comprises one or more barcode sequences; and a combination thereof.

A target sequence for a detectably labeled probe disclosed herein (e.g., a barcode sequence or complement thereof comprised in a probe or a product thereof) may be comprised in or associated with any analyte disclose herein, including an endogenous analyte (e.g., a viral or cellular nucleic acid), a labelling agent, or a product of an endogenous analyte and/or a labelling agent.

In some embodiments described herein, the analyte comprises or is associated with a target sequence. In some embodiments, a target sequence for a nucleic acid probe described herein is a marker sequence for a given analyte. A marker sequence is a sequence that identifies a given analyte (e.g., alone or in combination with one or more other marker sequences). Thus, in some embodiments, a marker sequence for a given target analyte is specific to that analyte, or unique, such that multiple target analytes can be distinguished from each other.

A “marker sequence” includes a sequence which marks, is associated with, or identifies a given analyte. It is a sequence by which a given analyte may be detected and distinguished from other analytes. Where an “analyte” comprises a group of related molecules e.g. isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g. a particular variant. In this way different variants, or isoforms, or mutants may be identified or distinguished from one another.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte or labelling agent (e.g., any of products described in Section V-B(iii)) as a tag or identifier (ID) sequence (e.g. a barcode) for the analyte or labelling agent. It may thus be a synthetic or artificial sequence.

In some embodiments, an endogenous analyte, labelling agent, or a product of an analyte or labelling agent may comprise multiple copies of the target sequence. For example, a probe molecule, or probe component, may comprise multiple copies of a target sequence. In another example, an amplification product may be generated which comprises multiple copies of the target sequence (e.g., multiple copies of a barcode sequence).

It will be understood that in the case of an analyte, product, or labelling agent comprising multiple target sequences, while each of the target sequences may comprise a binding site for a detectably labeled probe described herein, in practice not all of these binding sites may (or will) be occupied by a detectably labeled probe after hybridization. In some embodiments, it suffices that a number, or multiplicity, of such binding sites are bound by a detectably labeled probe. Thus, in some embodiments the detectably labeled probe may hybridize to at least one target sequence present in an analyte, labelling agent, or product of an analyte or labelling agent. In some embodiments, the detectably labeled probe hybridizes to multiple target sequences present in the analyte, labelling agent, or product of an analyte or labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, sequence analysis of nucleic acids (e.g., nucleic acids such as probes or RCA products comprising barcode sequences) can be performed by sequential hybridization (e.g., sequencing by hybridization and/or sequential in situ fluorescence hybridization). Sequential fluorescence hybridization can involve sequential hybridization of detectably labeled probes comprising an oligonucleotide and a detectable label. In some embodiments, a method disclosed herein comprises sequential hybridization of the detectably labeled probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectably labeled probes are described in US 2019/0161796, US 2020/0224244, US 2022/0010358, US 2021/0340618, and WO 2021/138676, all of which are incorporated herein by reference. In some embodiments, the methods provided herein can include analyzing the identifier sequences (e.g., analyte sequences or barcode sequences) by sequential hybridization and detection with a plurality of labeled probes (e.g., detection oligonucleotides).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

Also provided herein are detectable labels (e.g., of the detectably labeled probes) for use in the provided compositions and methods. Any suitable detectable label may be used. Detectable labels may have different detectable properties, including excitation and emission spectra, and emission lifetimes. In particular, detectable labels with different fluorescence emission lifetimes are contemplated for use with multi-cycle decoding with time-gated detection, including for use in the in situ detection of analytes. In some aspects, the provided methods comprise imaging a signal generated from a detectably labeled probe comprising a detectable label, such as a detectable label of the detectably labeled probes for detecting multiple analytes. In some aspects, detectable labels may be selected for use in multi-cycle decoding with time-gated detection. In some aspects the detectable labels comprise fluorescent moieties, such as fluorophores (e.g., GFP, RFP, Alexa dyes, Cy5, etc.). In some aspects, the detectable labels may comprise fluorophores with different and/or extended emission lifetimes, such as thermally activated delayed fluorescence (TADF) emitters or phosphorescent emitters, as described herein.

In some embodiments, a detectably labeled probe containing a detectable label can be used to detect one or more polynucleotide(s), probes (e.g., primary probes, intermediate probes), or products thereof (e.g. RCA products) described herein. In some embodiments, the methods involve incubating the detectably labeled probe containing the detectable label with the sample, washing unbound detectably labeled probe, and detecting the label, e.g., by imaging.

In some embodiments, the detectable label can be measured and quantitated. The terms “label” and “detectable label” comprise a directly or indirectly detectable moiety that is associated with (e.g., conjugated to) a molecule to be detected, e.g., a detectably labeled probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the detectable label is a fluorophore. In some aspects, the term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREEN™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345, incorporated herein by reference in its entirety for all purposes.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

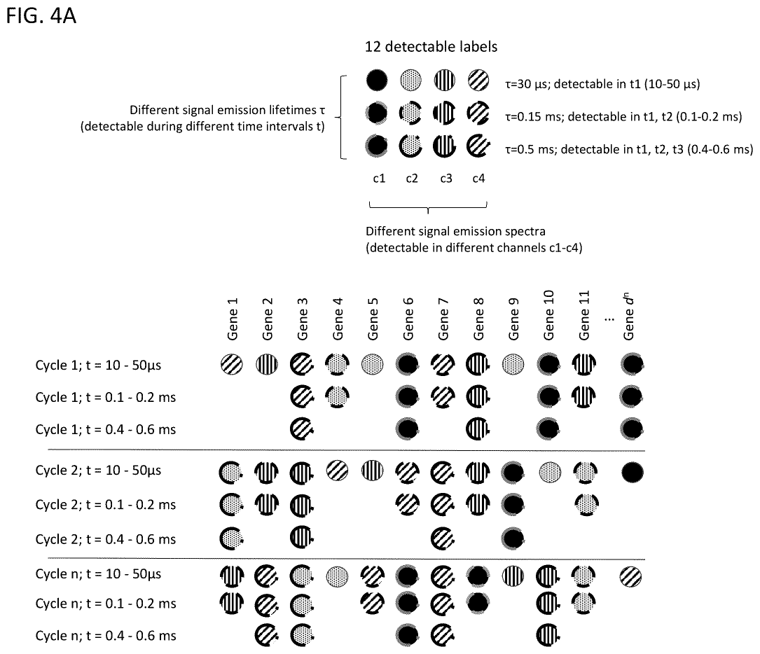

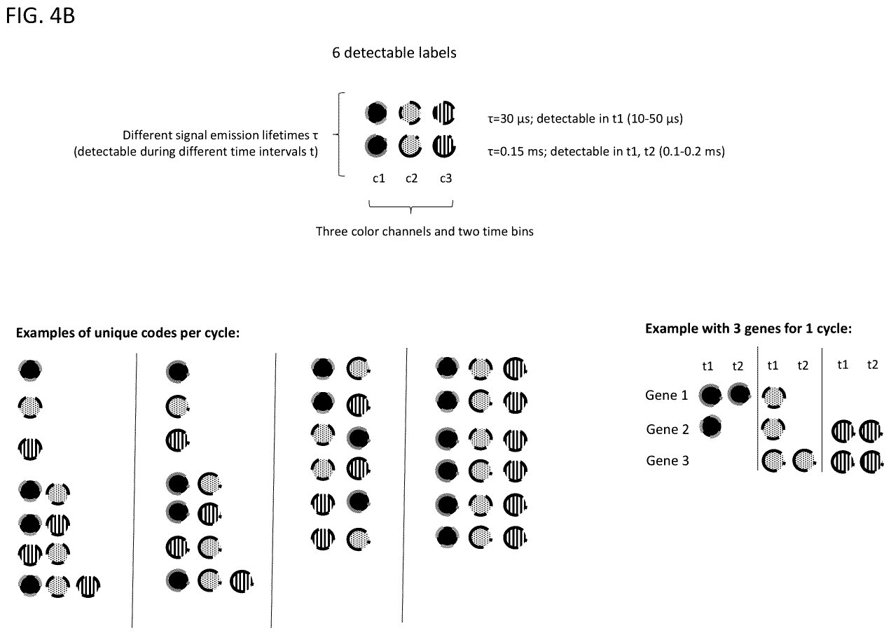

In some embodiments, one or more detectably labeled probes comprises an emitter, such as a thermally activated delayed fluorescence (TADF) emitter, and/or a phosphorescent emitter. In some embodiments, a detectably labeled probe comprises two or more different emitters, such two or more different thermally activated delayed fluorescence (TADF) emitters and/or phosphorescent emitters. In some cases, the two or more different emitters have different emission spectra. In some embodiments, multi-colored detectably labeled probes can encode higher information density than using a single color. For example, FIG. 4B shows the unique codes per cycle that can be used with 6 detectable labels that can be detected with three color channels and two time bins.

In some embodiments, one or more detectably labeled probes comprises a thermally activated delayed fluorescence (TADF) emitter. In some aspects, TADF emitters can have a longer fluorescent emission lifetime than traditional fluorescent emitters. In some aspects, different TADF emitters have different emission spectra and/or signal emission lifetimes.

In some embodiments, one or more detectably labeled probes comprises a phosphorescent emitter. In some aspects, phosphorescent emitters can have a longer fluorescent emission lifetime than traditional fluorescent emitters. In some aspects, different phosphorescent emitters have different emission spectra and/or signal emission lifetimes. In some cases, the phosphorescent emitter comprises an inorganic phosphor. In some embodiments, the inorganic phosphors may have a luminescence lifetime greater than 10 microseconds. In some embodiments, the phosphorescent emitter comprises strontium aluminate.

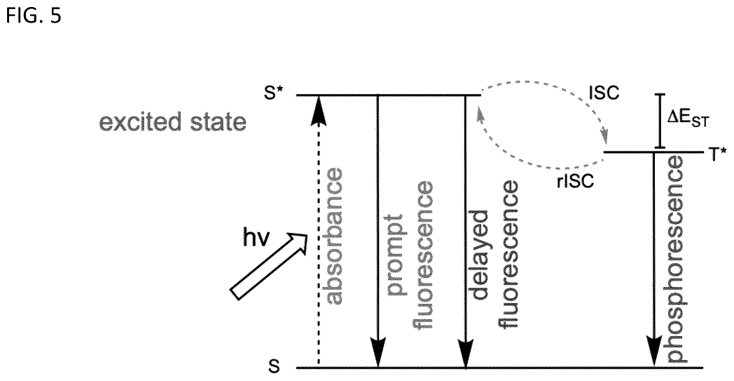

In some aspects, TADF emitters can have a longer fluorescent emission lifetime than traditional fluorescent emitters because TADF emitters undergo additional photophysical events in the excited state. In an exemplary photophysical pathway of a traditional fluorophore, an electron of the fluorophore is excited from the ground state (S) to a first singlet excited state (S*) upon absorption of a photon. The electron then returns to S and emits the photon to produce a fluorescent signal. As illustrated in FIG. 5, in an exemplary photophysical pathway of a TADF emitter, after excitation of an electron from S to S*, the electron can return to S and emit the photon to produce a fluorescence signal, as in a traditional fluorophore. Alternatively, the electron can also transfer to a first triplet excited state (T*) via intersystem crossing. From T*, the electron can relax to S by emitting a photon to produce a phosphorescent signal, or the electron can undergo reverse intersystem crossing to S*, and then return to S by emitting the photon to produce a delayed fluorescent signal. These additional photophysical steps can contribute to the extended fluorescence lifetimes of TADFs. In some aspects, phosphorescent emitters can have a longer fluorescent emission lifetime than traditional fluorescent emitters because phosphorescent emitters undergo additional photophysical events in the excited state. As illustrated in FIG. 5 in the exemplary photophysical pathway, phosphorescence is the result of emission with a change in spin (T* to S).

In some embodiments, a portion of the TADF emitters will absorb a photon and emit from the singlet state without undergoing conversion to the triplet state, resulting in prompt fluorescence with a lifetime of less than 5 ns. In some embodiments, a portion of the TADF emitters will absorb a photon, undergo intersystem crossing to the triplet state, and emit from the triplet state, resulting in phosphorescence. In some embodiments, a portion of the TADF emitters will absorb a photon and undergo non-radiative relaxation from the singlet state. In some embodiments, a portion of the TADF emitters will absorb a photon, undergo intersystem crossing to the triplet state, and undergo non-radiative relaxation from the triplet state.

In some embodiments, an emitter disclosed herein is a phosphorescence emitter. While TADF is a result of delayed emission without a change in spin (S1 to S0), phosphorescence is the result of emission with a change in spin (T1 to S0).

In some aspects, the emitter (e.g., TADF or phosphorescent emitter) can be susceptible to quenching by oxygen, for example when in a triplet state. In some embodiments, when a detectably labeled probe comprising an emitter (e.g., TADF or phosphorescent emitter) is used, the method is performed using a buffer comprising an oxygen scavenger. In some embodiments, the buffer comprises at least one enzymatic oxygen scavenger. In some embodiments, the buffer comprises PCA, PCD, rPCO, or a combination thereof. In some embodiments, the buffer comprises an enzyme for which the substrate is PCA or a recombinant enzyme thereof. In some embodiments, the buffer comprises PCA and PCD. In some embodiments, the buffer comprises PCA and rPCO. In some embodiments, the buffer comprises lactate oxyrase, glucose oxidase (GOx) or catalase, glucose pyranose oxidase (P2Ox), glucose, 6-hydroxy-2,5,7,8-tetramethylchroman-2-carboxylic acid (Trolox) or a combination thereof. In some embodiments, buffer comprises at least one non-enzymatic oxygen scavenger. In some embodiments, the buffer comprises sodium sulfite. In some embodiments, the buffer further comprises at least one protective agent. In some embodiments, the buffer comprises DABCO. In some embodiments, DABCO protects fluorophores from the triplet “dark state” by bringing them into the singlet ground state. In some embodiments, DABCO assists in maintaining molecular oxygen in the triplet ground state. In some embodiments, the buffer comprises Trolox, MV/AA, NAC/MV, propyl gallate, or a combination thereof, where MV is methyl viologen (also known as paraquat dichloride), AA is ascorbic acid, and NAC is N-acetyl-L-cysteine. In some embodiments, NAC confers strong antioxidant effects. In some embodiments, NAC reduces free radicals. In some embodiments, the buffer comprising an oxygen scavenger allows emission to persist in situ. In some embodiments, the emitter (e.g., TADF or phosphorescent emitter) is not encapsulated in a polymer matrix.

In some embodiments, the TADF or phosphorescent emitter has an emission profile with at least one emission peak with an emission maximum between about 400 nm and 700 nm, between about 400 nm and 600 nm, between about 400 nm and 500 nm, between about 500 nm and 700 nm, between about 500 nm and 600 nm, or between about 600 nm and 700 nm, and full-width half-maximum of at least about 50 nm, at least about 75 nm or at least about 100 nm. In certain embodiments, the TADF emitter or phosphorescent emitter has an emission profile with at least one emission peak with an absorption maximum between about 400 nm and 700 nm and full-width half-maximum of at least about 100 nm. In some embodiments, the TADF emitter or phosphorescent emitter has an emission profile with at least one absorption peak with an emission maximum between about 500 and about 600 nm and full-width half-maximum of at least about 100 nm.

In some aspects, the emission lifetime of a photoluminescent emitter can be defined by a decay curve according to the equation τ=1/(kᵣ+kₙᵣ), where τ is emission lifetime, kᵣ is radiative decay, and kₙᵣ is non-radiative decay. In some aspects, emission lifetime is the measure of the length of time that an emitter molecule remains in an excited state. In some aspects, no appreciable amount of photons are released after the emission lifetime.

In some aspects, emission lifetime refers to the amount of time that a detectable label provided herein, such as an emitter, emits a signal that is detectable experimentally or that meets a detection threshold, as defined by a user. In some aspects, emission lifetime is substantially shorter than the length of the total decay curve for an emitter (e.g., the signal is detectable for a substantially shorter amount of time than the total decay curve).

In some aspects, emission lifetime is the length of time between a) the offset of the stimulus for the detectable label, and b) the time at which the detectable label no longer emits a detectable signal in response to the stimulus. In some aspects, the signal emission lifetime of each detectable label is independently selected from: less than about 10 μs, between about 10 μs and about 100 μs, between about 100 μs and about 300 μs, between about 300 μs and about 1 ms, and greater than about 1 ms.

In some embodiments, the emitter (e.g., TADF or phosphorescent emitter) comprises at least one carbazole, phenoxazine, diphenylamine, triphenylamine, acridine, phenothiazine, diphenylacridine, phenazine, spiroacridine, or dimethylacridine moiety, each of which is optionally substituted with C₁-C₆ alkyl or C₁-C₆ alkoxy. In some embodiments, the emitter (e.g., TADF or phosphorescent emitter) comprises one to four carbazole, phenoxazine, diphenylamine, triphenylamine, acridine, or dimethylacridine moieties, each of which is optionally substituted with C₁-C₆ alkyl or C₁-C₆ alkoxy.

In some embodiments, the emitter (e.g., a TADF or phosphorescent emitter) comprises at least one cyanobenzene, dicyanobenzene, diphenyltriazine, diphenylsulfone, naphthalimide, heptazine, triazine, dicyanoimidazole, curcuminoid, oxadiazole, benzothiadiazole, pyrimidine, phenylbenzimidazole, dibenzodipyridophenazine, triarylboron, or dicyanopyrazino phenanthrene moiety. In some embodiments, the emitter (e.g., a TADF or phosphorescent emitter) comprises an organoboron moiety.

In some embodiments, the emitter comprises 2,4,5,6-Tetra(9H-carbazol-9-yl)isophthalonitrile (4CzIPN), 4-(3,6-Di-tert-butyl-9H-carbazole-9-yl)phenyl 3-(3,6-di-tert-butyl-9H-carbazole-9-yl)phenyl sulfone (DTC-DPS), 10,10′-(4,4′-Sulfonylbis(4,1-phenylene))bis(9,9-dimethyl-9,10-dihydroacridine) (DMAC-DPS), N-(4-Tert-butylphenyl)-1,8-naphthalimide-9,9-dimethyl-9,10-dihydroacridine (NAI-DMAC), or 7,10 bis(diphenylamino)dibenzo[f,h] quinoxaline-2,3-dicarbonitrile (DPA-DCPP).

In some embodiments, the emitter is a multi-resonance TADF emitter. In some embodiments, the multi-resonance TADF emitter has a smaller full width half maximum than emitters with traditional donor-acceptor frameworks. In some embodiments, the smaller full width half maximum may result in higher color purity. In some embodiments, the smaller full width half maximum may result in reduced cross talk between detection channels. In some embodiments, the smaller full width half maximum may result in higher color purity and reduced cross talk between detection channels. In some embodiments, the multi-resonance TADF emitter is 5,9-diphenyl-5,9-dihydro-5,9-diaza-13b-boranaphtho[3,2,1-de]anthracene (DABNA-1).

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

In some embodiments, an emitter (e.g., a TADF or phosphorescent emitter) comprised by a detectable label comprises one or more of any of the moieties selected from: DABNA-1; DABNA-2; DTC-DPS; DMAC-DPS; 4CzIPN; NAI-DMAC; DPA-DCPP; an organoboron moiety; a cyanobenzene moiety; a dicyanobenzene moiety; a diphenyltriazine moiety; a diphenylsulfone moiety; a naphthalimide moiety; a heptazine moiety; a triazine moiety; a dicyanopyrazino moiety; a phenanthrene moiety; a carbazole moiety optionally substituted with C₁-C₆ alkyl; a phenoxazine moiety optionally substituted with C₁-C₆ alkyl; a triphenylamine moiety optionally substituted with C₁-C₆ alkyl; a diphenylamide moiety optionally substituted with C₁-C₆ alkyl; an acridine moiety optionally substituted with C₁-C₆ alkyl; and a dimethylacridine moiety optionally substituted with C₁-C₆ alkyl.

In some embodiments, the emitter (e.g., TADF emitter or phosphorescent emitter) is encapsulated in a polymer matrix, optionally forming a polymer dot and/or an organic dot. In some embodiments, the polymer matrix comprises a semiconducting polymer. In some embodiments, the semiconducting polymer harvests energy from photons and transfers energy to the TADF emitter. In some embodiments, the emitter encapsulated in a semiconducting polymer, in comparison to the same emitter not encapsulated in a semiconducting polymer, exhibits reduced variance in emission lifetime, reduced oxygen quenching, increased brightness, increased photostability, increased molar absorptivity, and/or increased quantum yield. In some embodiments, the polymer matrix comprises a water-soluble polymer. In some embodiments, the polymer matrix allows for specific biological targeting. In some embodiments, each polymer dot or organic dot may comprise at least two different emitters (e.g., TADF emitters and/or phosphorescent emitters).

In some embodiments, the thermally activated delayed fluorescent (TADF) emitter or phosphorescent emitter is functionalized prior to being conjugating to an agent. In some embodiments, the TADF emitter or phosphorescent emitter is functionalized with a polynucleotide, optionally through a C₁-C₆ alkyl linker, or a polyethylene glycol linker.

FIG. 6 shows chemical structures of exemplary emitters of which the name, λₘₐₓ (lambda max; wavelength of strongest photon absorption), and τ (tau; fluorescence lifetime) are indicated.

In some embodiments, the detectable label may comprise any suitable combination of different types of photoluminescent moieties. In some embodiments, the detectable label may comprise any suitable photoluminescent moiety, such as a TADF, phosphorescent emitter, polymer dot, quantum dot, polymer nanoparticle, and/or organic dot, for example as described in Yu et al., 2017, Anal. Chem., 89(1):42-56; Gupta et al., 2019, Anal. Chem., 91(17):10955-10960; Wu and Chiu, 2013, Angew. Chem. Int. Ed. Engl., 52(11):3086-3109; Feng et al., 2013, Chem. Soc. Rev., 42(16):6620-6633; Tsuchiya et al., 2019, Chem. Comm., 55(36):5215-5218; Wu et al., ACS Nano., 2008, 2(11):2415-2423; Chang et al., Small, 2014, 10(21):4270-4275; Vu et al., Nat. Commun., 2022, 13(1):169, each of which is incorporated herein by reference in its entirety for all purposes.

### III. Multi-Cycle Decoding with Time-Gated Detection

In some embodiments, a method disclosed herein comprises performing multiple cycles of decoding using a plurality of detectably labeled probes, such as a library of probes (e.g., using the probes in a pre-determined order), wherein each cycle comprises contacting the analytes (e.g., any of analytes or products described in Section V-B, e.g., a signal amplification product such as rolling circle amplification products) with the probes, allowing the probes to directly or indirectly bind to their respective analytes, and detecting signals from the detectably labeled probes which have directly or indirectly bound to the analytes at one or more locations.

The methods herein have particular applicability in the detection of identifier sequences (e.g., analyte sequences or barcode sequences) in situ in a biological sample, including those using sequential cycles of detectably labeled probe hybridization to decode the identifier sequences.

In some embodiments, an identifier sequence herein comprises an analyte sequence, an analyte-derived sequence, or a complement thereof. In some embodiments, the analyte comprises a nucleic acid sequence, and an identifier sequence comprises the nucleic acid sequence in the analyte or a complement of the nucleic acid sequence.

In some embodiments, provided herein is a method comprising contacting a cell or tissue sample with a plurality of probes, each directly or indirectly binds to a different analyte in the sample. In some embodiments, each probe can comprise: i) a hybridization sequence for direct or indirect binding to its corresponding analyte and ii) one or more identifier sequences that are associated with, correspond to, and/or identify the corresponding analyte. In some embodiments, the one or more identifier sequences are not derived from the corresponding analyte but are barcode sequences assigned to the corresponding analyte.

In some embodiments, the signals are used to generate detectable signals (e.g., fluorescent or phosphorescent signals) in sequential cycles to identify one or more analytes at one or more locations. In some aspects, a signal code corresponding to a specific detectable label is assigned to the one or more locations in the biological sample where the detectable label is detected. In some aspects, one or more signal codes assigned to the same location over multiple cycles delineate a signal code sequence. In some aspects, the signal code sequence corresponds to a specific analyte in the sample. Thus, in some aspects, a signal code sequence reveals the identity of a specific analyte in a specific location in the biological sample.

In some embodiments, a plurality of analytes can be decoded (e.g., identified or distinguished from other analytes) by contacting a sample containing or suspected of containing the analyte with detectably labeled probes that recognize barcode regions of primary probes bound to the analytes or products thereof, to produce probe binding patterns in sequential probe binding cycles, whereby patterns of detectably labeled probe binding can be compared to a list of known and/or allowed order of signal codes (e.g., identifiers) corresponding to the analytes (e.g., a codebook or “whitelist”). In some embodiments, the list of known order of signal codes (e.g., identifiers) corresponding to the analytes includes one or more dark cycles.

In some embodiments, decoding comprises compiling, processing, and analyzing a plurality of optical signals detected from sequential cycles at a location in a biological sample. In some aspects, a location comprising optical signals comprises optical signals from at least two neighboring spots (e.g., two analytes, such as two RCPs) that are close in proximity (e.g., optically overlapping). In some embodiments, a location may comprise two or more neighboring spots that are within 1 μm distance. In some embodiments, a location may comprise two or more neighboring spots that are within about 0.5 to 1 μm distance. In some cases, each location including all spots (e.g., overlapping spots) within that distance is observed and potential signal sequence chains are generated for this location. In some aspects, the optical signals from neighboring spots are detected and processed to determine which optical signals are associated with the first of the neighboring spots vs. the optical signals that are associated with the second of the neighboring spots. In some cases, an optical signal can be a presence of a signal (e.g., a detected signal in a channel) or an absence of a signal in the location. In some cases, the processing includes compiling the detected optical signals. In some cases, the processing includes comparing observed optical signals from various sequential cycles of detection to a list of known order of signal codes (e.g., of the identifiers) corresponding to the analytes (e.g., from a codebook or “whitelist”). In some cases, optical signals corresponding to the analyte are processed.

In some embodiments, provided herein is a method for nucleic acid sequence detection, comprising: (a) contacting a sample with multiple sets of detectably labeled probes in sequential cycles, wherein each set is specific for a corresponding target nucleic acid sequence and each set comprises multiple probes that (i) hybridize to a particular nucleic acid sequence and (ii) are directly or indirectly labeled with a detectable label which may be the same or different for probes in the same probe set, wherein the sequential cycles comprise contacting the sample with probes within a set in a pre-determined order which corresponds to a signal code sequence for the corresponding target nucleic acid sequence; and (b) obtaining a signal code sequence for each target nucleic acid sequence, wherein the signal code sequence comprises signal codes corresponding to the signals or absence thereof from detectable labels for probes in the sequential cycles, thereby detecting the target nucleic acid sequences in the sample.

In some embodiments, described herein is a method of localized detection of multiple target nucleic acid sequences in a sample, wherein each target nucleic acid sequence is detected using a circularizable primary probe specific for said target sequence which is circularized upon hybridization to the target sequence and is amplified by rolling circle amplification (RCA) to produce a rolling circle product (RCP), each circularizable primary probe comprising a different barcode region specific for a different target nucleic acid sequence, and each RCP containing multiple complementary copies of the barcode region, wherein the barcode region is decoded in multiple sequential decoding cycles each using hybridization probes (e.g., intermediate probes) which hybridize to the complementary copies of the barcode region in an RCP and allow detectable signals to be generated which together yield a unique signal code sequence which identifies the target nucleic acid sequence.

In some embodiments, described herein is a method of localized detection of multiple target nucleic acid sequences in a sample, wherein each target nucleic acid sequence is detected using a linear primary probe specific for said target sequence. The linear primary probe may comprise one or more overhang regions which does not hybridize to the target sequence. The overhang region of the primary probe may comprise one or barcode regions specific for a different target nucleic acid sequence. In some instances, an amplification product may be formed using the primary probe and the amplification product may comprise multiple copies of the barcode region, wherein the barcode region is decoded in multiple sequential decoding cycles each using hybridization probes (e.g., intermediate probes) which hybridize to the complementary copies of the barcode region in the amplification product and allow detectable signals to be generated which together yield a unique signal code sequence which identifies the target nucleic acid sequence.

In some embodiments, a sequential decoding scheme involves detecting repeated signals from a given target in multiple cycles, and the target may be in the same position in the sample in the different cycles. In some embodiments, a method disclosed herein comprises the localized detection of the target nucleic acid sequences. In some embodiments, the target nucleic acid sequence is present at a fixed or defined location in the sample, and is detected at that location. The target nucleic acid sequence may be localized by virtue of being present in situ at its native location in the sample (e.g. a cell or tissue sample), or of being attached or otherwise localized to a target analyte which is present in situ at its native location in the sample. The target nucleic acid sequence may be immobilized in the sample. Thus, a target analyte or a target nucleic acid sequence may be artificially immobilized in the sample, e.g. fixed on or to a solid surface, or bound to an immobilizing moiety provided on a solid surface.

In some embodiments, the number of decoding cycles that are required to fully identify all of the individual signal codes which make up the signal code sequence for all of the target nucleic acid sequences is greater than the number of individual signal codes which make up the signal code sequences. In some embodiments, the number of decoding cycles required to fully identify all of the signal code sequences is greater than the number of hybridization probes in at least one set of hybridization probes, or greater than the number of signal code positions in a least one signal code sequence. Generally speaking the signal code sequences for different targets may be designed to be of the same length, but this is not essential.

In some embodiments, provided herein is a method comprising detecting target nucleic acid sequences by primary probes, circularizable probes or probe sets, or wherein the primary probe is detected by detecting a rolling circle amplification (RCA) product of the primary probe.

In some embodiments, each target nucleic acid sequence in the sample is detected using a circularizable probe specific for that target sequence which is circularized upon hybridization to the target sequence and is amplified by rolling circle amplification (RCA) to produce a rolling circle product (RCP). In some embodiments, each circularizable probe comprises a different barcode region specific for a different target nucleic acid sequence, and therefore each RCP contains multiple complementary copies of the barcode region.

In some embodiments, the barcode region is decoded in multiple sequential decoding cycles. In some embodiments, the barcode region corresponds to a unique signal code sequence which is specific to the target nucleic acid sequence. In some embodiments, each decoding cycle uses hybridization probes (e.g., intermediate probes) which hybridize to the complementary copies of the barcode region of the probe or a product thereof and comprise regions for generating detectable signals (e.g. decoding regions), wherein each signal corresponds to an individual signal code, and the signal codes together yield the unique signal code sequence which identifies the target nucleic acid sequence.

In some embodiments, image registration is performed. In some aspects, image registration comprises aligning signals and/or images obtained from various cycles onto a common coordinate system. When obtaining images or detecting signals from a sample across multiple cycles, the sample or imaging apparatus may shift, causing an offset of images from one cycle to the next. In some aspects, image registration compensates for these shifts, allowing the user to identify the same relative location within the sample between different images, and/or overlay images that are spatially aligned. In some embodiments, one or more of the detected signals are used for image registration. In some embodiments, “spot-calling” signals (e.g., signals generated that correspond to the locations of all or a subset of the analytes in a sample) are used for image registration. In some embodiments, spot-calling signals provide a plurality of physical landmarks within the sample that can be used to align multiple images. In some embodiments, signal codes can be assigned to locations associated with spot-calling signals. In some embodiments, image registration allows signal codes from multiple cycles to be assigned to the same location (e.g. a location associated with a spot-calling signal), allowing a signal code sequence to be constructed for that location. In some embodiments, image registration is performed using computational methods. In some embodiments, image registration is performed manually, guided, or adjusted by a user.

In some aspects, one or more detectable labels are detected using time-gated detection of signals from the detectable labels. In some aspects, the detectable labels emit a signal (e.g., fluorescence or phosphorescence) following a stimulus (e.g., light). In some aspects, different detectable labels have different signal emission lifetimes. In some aspects, the signal emission lifetime of a detectable label is the amount of time that the detectable label emits a detectable signal after the emission-inducing stimulus (e.g., the amount of time that detectable fluorescence is emitted from a fluorophore after the stimulation with light is terminated). In some aspects, a detectable label can be identified or distinguished from other detectable labels based on signal emission lifetime, and/or based on the channel (e.g., fluorescent color) in which the detectable label is detected.

In some embodiments, a method for analyzing a biological sample, comprising: a) contacting the biological sample with a plurality of first detectably labeled probes for detecting a first analyte and a second analyte, wherein each first detectably labeled probe is configured to directly or indirectly bind to a different analyte or product thereof and comprises a detectable label, b) detecting signals associated with the detectable labels of the plurality of first detectably labeled probes, or absence of the signals, at one or more locations in the biological sample, c) contacting the biological sample with a plurality of subsequent detectably labeled probes for detecting the first analyte and the second analyte, wherein each subsequent detectably labeled probe is configured to directly or indirectly bind to a different analyte or product thereof and comprises a detectable label, d) detecting signals associated with the detectable labels of the plurality of subsequent detectably labeled probes, or absence of the signals, at the one or more locations in the biological sample, wherein the detecting in b) and d) comprises detecting signals or absence thereof during a time interval t1 and during a time interval t2, respectively, wherein the onset of time interval t2 is later than the onset of time interval t1, wherein at least one detectable label of the plurality of first detectably labeled probes and/or the plurality of subsequent detectably labeled probes is detectable during time interval t1 and not detectable during time interval t2, wherein at least one detectable label of the plurality of first detectably labeled probes and/or the plurality of subsequent detectably labeled probes is detectable during time interval t2, wherein signal codes corresponding to the signals or absence thereof detected in b) and d) during time interval t1 and t2 are used to generate a first signal code sequence corresponding to the first analyte and a second signal code sequence corresponding to the second analyte, thereby identifying the first analyte and the second analyte at the one or more locations in the biological sample.

In some embodiments, at least one signal is detected that is associated with a detectable label of the plurality of first detectably labeled probes and/or plurality of subsequent detectably labeled probes is detected during time interval t2.

In some aspects, a detectable label can be identified or distinguished from other detectable labels based on signal emission lifetime. In some aspects, signals or absence thereof are detected during two or more detection time intervals with respect to stimulation, and a detectable label is identified based on the presence or absence of a signal detected in each detection time interval. In an exemplary embodiment, a first detectable label and second detectable label are identified in different locations of a biological a sample using time-gated detection. The first detectable label has a signal emission lifetime of τ=0.2 ms and the second detectable label has a signal emission lifetime of τ=0.5 ms. Following stimulation of both detectable labels, detection is performed during a first detection time interval t1 (0.1-0.2 ms) and a second detection time interval t2 (0.4-0.5 ms). The first detectable label is detectable during t1 but not t2, whereas the second detectable label is detectable during both t1 and t2. Thus, in a location with a signal detected at t1 but not t2, the first detectable label is identified. In a location with a signal detected at both t1 and t2, the second detectable label is identified. In some embodiments, the first detection time interval t1 and the second detection time interval t2 are non-overlapping (e.g., temporally distinct). In some embodiments, the first detection time interval t1 and the second detection time interval t2 are overlapping (e.g., the onset of t2 is before the end of t1).

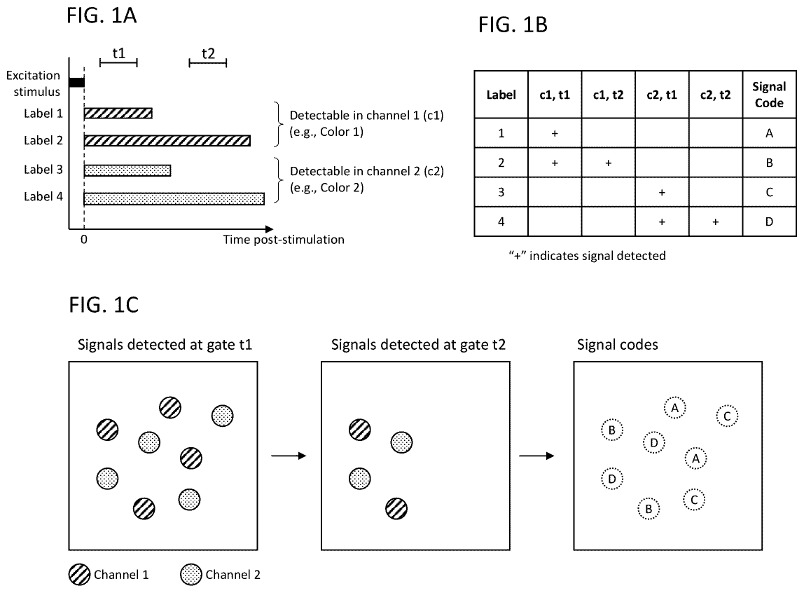

Time-gated detection can be further illustrated with reference to the drawings. FIGS. 1A-1C show schematics illustrating exemplary time-gated detection of detectable labels with different signal emission lifetimes. As shown in FIG. 1A, an excitation stimulus (e.g. light) causes the labels to emit a detectable signal, such as a fluorescent signal. The time during which the signal is detectable is represented by the length of the corresponding bars for each label. The signals are detectable for different amounts of time post-stimulation (e.g., the detectable labels have different signal emission lifetimes.) Label 1 and label 2 are each detectable in a first detection channel (c1) for different amounts of time post-stimulation. Signals are detected in channel c1 during a first detection time interval t1 and a second detection time interval t2. Label 1 is detected during t1 but not t2, whereas label 2 is detected during t1 and t2. Similarly, label 3 and label 4 are each detectable in a second detection channel (c2). Label 3 is detected during t1 but not t2, whereas label 4 is detected during t1 and t2. Thus, each of the four labels are detected in a different combination of channels and detection time intervals, which correspond to a signal code for each detectable label, as shown in FIG. 1B. FIG. 1C shows a schematic illustrating an exemplary decoding cycle. Each square represents the same area of an imaged biological sample. The biological sample is contacted with a plurality of detectably labeled probes for detecting multiple analytes, wherein each detectably labeled probe is configured to bind to a different analyte or product thereof and comprises a detectable label. In the figure, the probes comprise the four detectable labels described above for FIG. 1A and FIG. 1B. Signals are detected in channels c1 and c2 during detection time intervals t1 (left) and t2 (middle). The signals and absence thereof are used to assign a signal code to each location where a signal was detected (right), corresponding to each detectable label.

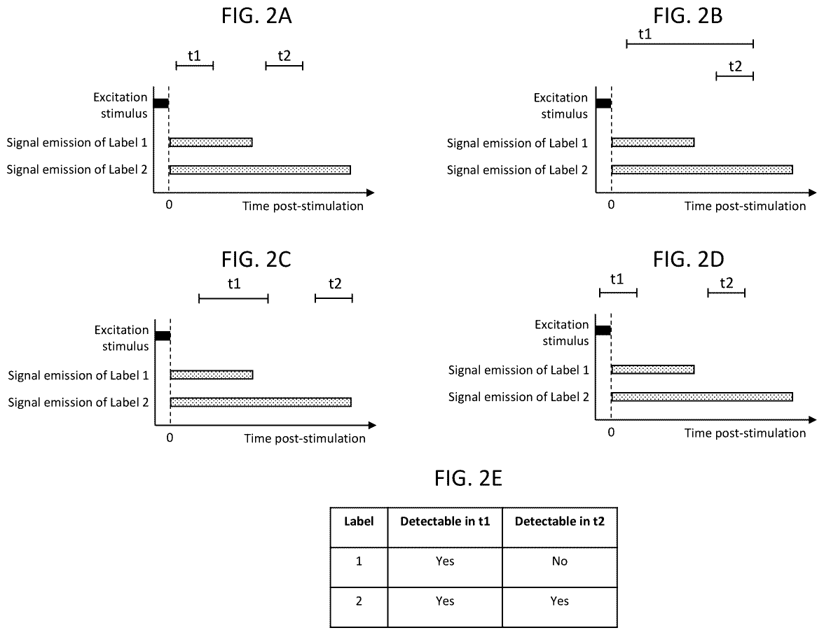

FIGS. 2A-2D show exemplary detection time intervals that can be used for time-gated detection and decoding of detectable labels. A first and second detectable label are excited, and detection is performed during a first detection time interval t1 and a second detection time interval t2. As summarized in the table (FIG. 2E), for each example, the first label is detected during t1 and not t2, whereas the second label is detected during t1 and t2. Time-gated detection thereby allows for identification of the different detectable labels, even if they are detected in the same channel. A signal may be detected during any portion of a detection time interval. For example, in some embodiments, a signal is detected for the entire duration of the detection time interval (e.g., detection of label 1 during t1 and detection of label 2 during t2 in FIG. 2A). In some embodiments, a signal is detected for a portion of the detection time interval (e.g., detection of label 1 during t1 in FIG. 2A; detection of label 2 during t2 in FIG. 2C). All or a portion of a detection time interval may occur during an excitation stimulus (e.g., t1 in FIG. 2D). In some embodiments, a detection time interval may occur during an excitation stimulus if the excitation and detection wavelengths are distinct (e.g., excitation at about 400 nm wavelength and detection at about 550 nm wavelength). In some embodiments, the excitation and detection wavelength of the detectable label are distinct. In some embodiments, the detection time intervals overlap (e.g., t1 and t2 in FIG. 2B). In all such configurations, the first and second detectable labels can be identified based on detection of a signal or absence thereof during the detection time intervals.

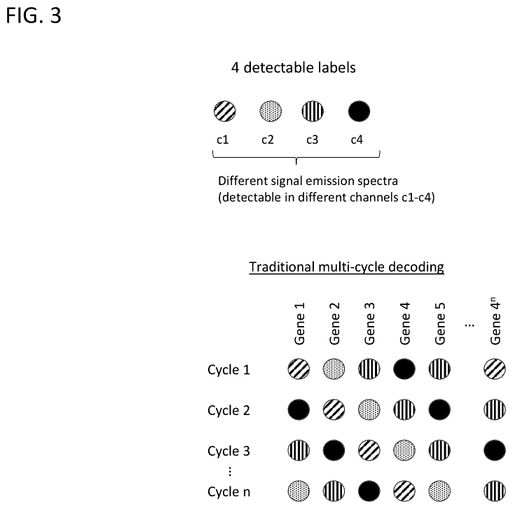

FIG. 3 shows a schematic illustrating exemplary “traditional” multi-cycle decoding of analytes using four detectable labels, each detected in a different channel (c1-c4) without time-gated detection. In each cycle, a biological sample is contacted with a plurality of detectably labeled probes for detecting multiple analytes (e.g., genes), wherein each detectably labeled probe is configured to bind to a different analyte or product thereof and comprises one of the four detectable labels. Signals corresponding to each detectable label are detected at one or more locations in the biological sample, and signal codes corresponding to the detectable labels are assigned to each location. Multiple cycles are performed with different combinations of detectable labels corresponding to each analyte. A signal code sequence is generated for one or more locations, each signal code sequence comprising the signal codes assigned to a given location in the biological sample over multiple cycles. The signal code sequence may be used to identify the presence of an analyte at one or more locations. An exemplary signal code sequence for Gene 1 comprises the sequence of signal codes assigned to the sequence of detectable labels shown in the column corresponding to Gene 1. In a decoding scheme with d detectable labels and n cycles, the number of genes that can be decoded is d\hat{ }n. Thus, in traditional four-color multi-cycle decoding, a maximum of 4\hat{ }n genes can be decoded in n cycles (e.g., 64 genes in 3 cycles).

FIG. 4A shows a schematic illustrating exemplary cycles of multi-cycle decoding of analytes using twelve detectable labels, which are identified based on detection of signals in four different channels during three different detection time intervals. In the panel showing the 12 detectable labels, detectable labels in different columns have different signal emission spectra (e.g., each column can represent a different fluorescent color), and detectable labels in each column can be detected in a different channel (c1-c4). Detectable labels in different rows have different signal emission lifetimes (τ=30 μs, τ=0.15 ms, and τ=0.5 ms), allowing them to be detected using time-gated detection, for instance, detectable labels of τ=30 μs are detectable during detection time interval t1=10-50 μs, detectable labels of τ=0.15 ms are detectable during detection time intervals t1=10-50 μs and t2=0.1-0.2 ms, and detectable labels of τ=0.5 ms are detectable during detection time intervals t1=10-50 t2=0.1-0.2 ms, and t3=0.4-0.6 ms. In each decoding cycle, detection is performed in the four channels (c1-c4) corresponding to the four groupings of signal emission spectra, and at 3 detection time intervals (t1-t3). In some instances, the upper limit of the number of analytes that can be decoded is d\hat{ }n, where d is the number of different detectable labels (e.g., 12 in the example shown in FIG. 4A) and n is the number of probe hybridization cycles. As in traditional multi-cycle decoding, signal codes corresponding to the detectable labels are assigned to their detected locations. However, whereas in traditional multi-cycle decoding, the signal code corresponds only to the channel of detection (e.g., signal color detected in the channel), in multi-cycle decoding with time-gated detection, the signal code corresponds both to the channel of detection and the detection time interval(s) of detection. As in traditional multi-cycle decoding, multiple cycles are performed with different combinations of detectable labels corresponding to each analyte. A signal code sequence is generated for one or more locations, each signal code sequence comprising the signal codes assigned to a given location in the biological sample over multiple cycles. The signal code sequence may be used to identify the presence of an analyte at one or more locations. In a decoding scheme with d detectable labels and n cycles, the number of genes that can be decoded is d\hat{ }n. In the exemplified multi-cycle decoding scheme with time-gated detection, a maximum of 12\hat{ }n genes can be decoded in n cycles (e.g., 1,728 genes in 3 cycles). Thus, it can be seen that in some aspects, time-gated detection greatly increases the maximum number of analytes (e.g., genes or transcripts thereof) that can be decoded for a given number of cycles. In some aspects, in order to decode a given number of analytes (e.g., genes or transcripts thereof), time-gated detection allows the use of a smaller number of probe hybridization and signal detection cycles, thereby significantly reducing the amount of time required to decode the analytes. In some instances, the use of multi-color detectably labeled probes can further increase the number of analytes that can be decoded by using at least two different colors per gene within a cycle (e.g., examples shown in FIG. 4B).

### IV. Signal Amplification, Detection, and Analysis

In some embodiments, the present disclosure relates to the detection of analytes (e.g., nucleic acids sequences) in situ using probe hybridization. In some embodiments, the detection of analytes comprises generation of amplified signals associated with the probes (e.g., primary probes).

In some embodiments, detection comprises an amplification product of one or more polynucleotides, for instance, a circular probe or circularizable probe or probe set hybridized to an analyte. In some embodiments, the amplifying is achieved by performing extension or amplification such as rolling circle amplification (RCA).

In some aspects, the provided methods involve analyzing, e.g., detecting or determining, one or more sequences present in a target nucleic acid, or probes directly or indirectly hybridized thereto. In some aspects, the provided methods involve analyzing one or more detectable signals associated with the target nucleic acid. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments, the detection may be spatial, e.g., in two or three dimensions. In some embodiments, the detection may be quantitative, e.g., the amount or concentration of detectable signals (and of a corresponding target nucleic acid) may be determined. In some embodiments, the primary probes, intermediate probes, higher order probes, and/or detectably labeled probes may comprise any of a variety of entities able to hybridize a nucleic acid, e.g., DNA, RNA, LNA, and/or PNA, etc., depending on the application.

Detectably labeled probes may comprise recognition sequences that hybridize to sequences in the probes forming a hybridization complex with the target nucleic acid (e.g. primary probes hybridized to analytes, RCA products thereof, and intermediate probes), for example to barcode sequences or complements thereof, or decoding regions. The recognition sequences may be of any length, and multiple recognition sequences in the same or different probes of the hybridization complex may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. In some embodiments, the one or more recognition sequences individually are between about 5 and about 50 nucleotides in length. Combinations of any of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof of a circular or circularized probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, a hybridized primary probe as described herein can be detected with a method of multi-cycle decoding with time-gated detection that comprises signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is incorporated herein by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is incorporated herein by reference in its entirety), e.g., primer exchange reactions such as signal amplification by exchange reaction (SABER) or SABER with DNA-Exchange (Exchange-SABER). In some embodiments, a non-enzymatic signal amplification method may be used.

In some embodiments, detection of a hybridized primary probe or product thereof as described herein includes an assembly for branched signal amplification. In some embodiments, the assembly complex comprises an amplifier hybridized directly or indirectly (via one or more oligonucleotides) to a sequence of the primary probe or product thereof. In some embodiments, the assembly includes one or more amplifiers each including an amplifier repeating sequence. In some aspects, the one or more amplifiers is labeled. For exemplary branched signal amplification, see e.g., U.S. Pat. Pub. No. US20200399689A1, US20220064697A1, and Xia et al., Multiplexed Detection of RNA using MERFISH and branched DNA amplification. Scientific Reports (2019), each of which is fully incorporated by reference herein.

In some embodiments, detection of hybridized primary probes or products thereof includes hybridization chain reaction (HCR). HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. Exemplary methods and compositions for LO-HCR are described in US 2021/0198723, incorporated herein by reference in its entirety.

In some embodiments, the primary probe or product thereof as described herein can be detected with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, an endogenous analyte, probe or product thereof may be contacted with a plurality of concatemer primers and a plurality of labeled probes. see e.g., U.S. Pat. Pub. No. US20190106733, the content of which is incorporated herein by reference in its entirety, for exemplary molecules and PER reaction components.

In some embodiments, a hybridized primary probe or product thereof as described herein can be detected by providing probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in an overhang region of the first and/or second probe).

In some embodiments, the methods comprise determining the sequence of all or a portion of the hybridized primary probe or product thereof, such as one or more barcode sequences present therein. In some embodiments, the analysis and/or sequence determination comprises sequencing all or a portion of the primary probe or product thereof, and/or in situ hybridization to the primary probe or product thereof. In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises directly or indirectly hybridizing to the primary probe or product thereof a detection oligonucleotide labeled with a fluorophore, an isotope, a mass tag, or a combination thereof. In some embodiments, the detection or determination comprises imaging the amplification product. In some embodiments, the target nucleic acid is an mRNA in a cell or tissue sample, and the detection or determination is performed when the target nucleic acid and/or the amplification product is in situ in the cell or tissue sample.

In some embodiments, the barcode regions or complements thereof comprised by the probes forming a hybridization complex with the target nucleic acid or products thereof are targeted directly or indirectly by detectably labeled detectable oligonucleotides, such as fluorescently labeled oligonucleotides. In some embodiments, one or more decoding schemes are used to decode the signals, such as fluorescence or phosphorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of detectably labeled probes as described herein for multi-cycle decoding with time-gated detection. Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entireties. In some embodiments, these assays enable signal amplification, combinatorial decoding, and error correction schemes at the same time.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent or phosphorescent product for imaging. In some aspects, the detecting comprises determining a signal, e.g., a fluorescent signal, using microscopy.

In some aspects, the detection (comprising imaging) is carried out using any suitable method of microscopy for capturing optical signals, such as fluorescence and phosphorescence signals. In some aspects, the detection is carried out using two-photon microscopy, confocal microscopy, epifluorescence microscopy, intact tissue expansion microscopy, and/or CLARITY™-optimized light sheet microscopy (COLM). The methods disclosed herein may be carried out in conjunction with any suitable additional method of microscopy for collecting information about the biological sample.

In some aspects, the detection comprises time-gated detection. Time-gated detection may be implemented by any suitable means. For example, in some embodiments, the detector is active for one or more detection time intervals. In other embodiments, physical gating is used, wherein a barrier shields the detector and is removed for detection for a period of time corresponding to a desired detection time interval. In some embodiments, the time-gating is defined by a user and implemented via software. In some embodiments, the time-gated detection is used to capture a signal during any suitable detection time interval, such as any detection time interval described herein that coincides with the signal emission lifetime of a detectable label.

In some embodiments, fluorescence microscopy is used for detection and imaging of the detectably labeled probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, such as an epifluorescence microscope or confocal microscope.

In some embodiments, confocal microscopy is used for detection and imaging of the detectably labeled probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

### V. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The biological sample can be obtained as a section of a cell pellet or a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (1) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 80, 90, or 100 μm or more. Typically, the thickness of a tissue section is between 1-100 μm, 1-μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe (e.g., padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a template for amplification. Alternatively, the specific binding partner may be coupled to a nucleic acid, which may be detected using an amplification strategy, e.g. in an assay which uses or generates an amplification product.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(1) Endogenous Analytes

In some embodiments, an analyte herein is endogenous to a biological sample and can include nucleic acid analytes and non-nucleic acid analytes. Methods and compositions disclosed herein for multi-cycle decoding with time-gated detection can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

(ii) Labelling Agents

In some embodiments, provided herein are methods and compositions for analyzing endogenous analytes (e.g., RNA, ssDNA, cell surface or intracellular proteins, and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. In some embodiments, an analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labelling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be a fluorophore, a radioisotope, a molecule capable of a colorimetric reaction, a magnetic particle, or any suitable fluorescent moiety, including thermally activated delayed fluorescence (TADF) emitter, polymer dot, quantum dot, polymer nanoparticle, and/or organic dot, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample using multi-cycle decoding with time-gated detection. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed. Methods and compositions disclosed herein for multi-cycle decoding with time-gated detection can be used to analyze nucleic acid analytes (e.g., using a nucleic acid probe or probe set that directly or indirectly hybridizes to products of an endogenous analyte and/or a labelling agent).

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

(b) Ligation

In some embodiments, a product of an endogenous analyte and/or a labelling agent is a ligation product. In some embodiments, the ligation product is formed between two or more endogenous analytes. In some embodiments, the ligation product is formed between two or more labelling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some embodiments, a primer extension reaction comprises any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination.

### VI. Compositions, Kits and Systems

In some embodiments, disclosed herein is a composition that comprises a plurality of detectably labeled probes each comprising a detectable label (e.g., including any of the fluorescent or phosphorescent molecules and materials described in Section II), wherein the detectable labels of the plurality of detectably labeled probes have different signal emission lifetimes.

In some aspects, provided herein is a kit for use in a method for analyzing a biological sample. In some aspects, the kit comprises a plurality of first detectably labeled probes for detecting a first analyte and a second analyte, wherein each first detectably labeled probe is configured to directly or indirectly bind to a different analyte or product thereof and comprises a detectable label. The plurality of first detectably labeled probes can be contacted with the biological sample. In some embodiments, the kit comprises reagents for detecting signals associated with the detectable labels of the plurality of first detectably labeled probes, or absence of the signals, at one or more locations in the biological sample. In some embodiments, the kit comprises a plurality of subsequent detectably labeled probes for detecting the first analyte and the second analyte, wherein each subsequent detectably labeled probe is configured to directly or indirectly bind to a different analyte or product thereof and comprises a detectable label. The plurality of subsequent detectably labeled probes can be contacted with the biological sample. In some embodiments, the kit comprises reagents for detecting signals associated with the detectable labels of the plurality of subsequent detectably labeled probes, or absence of the signals, at the one or more locations in the biological sample. In some embodiments, the detecting comprises detecting signals or absence thereof during a detection time interval t1 and during a detection time interval t2, respectively. In some embodiments, the onset of t2 is later than the onset of t1. In some embodiments, at least one detectable label of the plurality of first detectably labeled probes and/or the plurality of subsequent detectably labeled probes is detectable during t1 and not detectable during t2, and at least one detectable label of the plurality of first detectably labeled probes and/or the plurality of subsequent detectably labeled probes is detectable during t2. In some embodiments, signal codes corresponding to the signals or absence thereof detected during t1 and t2 are used to generate a first signal code sequence corresponding to the first analyte and a second signal code sequence corresponding to the second analyte, thereby identifying the first analyte and the second analyte at the one or more locations in the biological sample.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing (e.g., crosslinking), embedding, and/or permeabilizing the biological sample. In some embodiments, the kits contain reagents, such as enzymes and buffers. In some aspects, the kit can also comprise any of the reagents described herein, e.g., wash buffer. In some embodiments, the kits contain reagents for detection and/or sequencing, such as detectably labeled probes or detectable labels. In some embodiments, the kits optionally contain other components, for example nucleic acid primers, enzymes and reagents, buffers, nucleotides, and reagents for additional assays.

In some aspects, provided herein is a system comprising an opto-fluidic instrument. In some embodiments, the opto-fluidic instrument comprises, e.g., a fluidics module, an optical module, and a sample module. In some embodiments, the fluidics module is configured to deliver one or more reagents (e.g., detectably labeled probes conjugated to TADF emitters described in Section II) to the biological sample and/or remove spent reagents therefrom. Additionally, the optics module can be configured to illuminate the biological sample with light having one or more spectral emission curves (over a range of wavelengths) and subsequently capture one or more images of emitted light signals from the biological sample during one or more probing cycles each during one or more specified detection time intervals. In various embodiments, the captured images may be processed in real time and/or at a later time to determine the presence of the one or more target molecules in the biological sample, as well as three-dimensional position information associated with each detected analyte. Additionally, the opto-fluidics instrument can include a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

In some examples, an opto-fluidic instrument may also include a sample module configured to receive the sample, and an optics module including an imaging system for exciting the biological sample with a pulsed light source such that the biological sample emits a plurality of fluorescence photons; and detecting during one or more detection time intervals. The in-situ analysis system may also include other ancillary modules configured to facilitate the operation of the opto-fluidic instrument, such as, but not limited to, cooling systems, motion calibration systems, etc. The detection of a portion of the fluorescence photons during a detection time interval can be implemented by any suitable means. For example, in some embodiments, the detector of the opto-fluidic instrument is active for one or more detection time intervals. In other embodiments, physical gating is used, wherein a barrier shields the detector and is removed for detection for a period of time outside of a detection time interval. In some embodiments, the time-gating is defined by a user and implemented via software. In some embodiments, the time-gated detection is used to capture a signal during any suitable detection time interval.

### VII. Applications

In some aspects, the provided embodiments can be applied in an in situ method of analyzing nucleic acid sequences, such as fluorescent in situ hybridization (FISH)-based methods, in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to detect a signal associated with a detectable label of a nucleic acid probe that is hybridized to a target sequence of a target nucleic acid in a biological sample.

In some embodiments, the target nucleic acid comprises a single-nucleotide polymorphism (SNP). In some embodiments, the target nucleic acid comprises is a single-nucleotide variant (SNV). In some embodiments, the target nucleic acid comprises a single-nucleotide substitution. In some embodiments, the target nucleic acid comprises a point mutation. In some embodiments, the target nucleic acid comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences can become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using suitable techniques, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, or composition to be detected. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine 0-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, C₁-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (Di1C18(5)), DIDS, Dil (Di1C18(3)), DiO (DiOC18(3)), DiR (Di1C18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™1/PO-PRO™-1, POPO™3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families can provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the present disclosure.

### Example 1: Multi-Cycle Decoding Using Time-Gated Detection

This example demonstrates a method of using sequential hybridization of detectably-labeled probes to detect target analytes in a sample.

A biological sample is contacted with a library of primary probes, each primary probe comprising (i) a binding region configured to bind to a target analyte, and (ii) a barcode region associated with the analyte. Each primary probe for a target analyte has a different binding region and barcode region associated with each different target analyte. The primary probes comprise circularizable probes or probe sets (e.g. padlock probes). The primary probes are hybridized to target nucleic acid analytes and ligated to generate circularized primary probes. An RCA primer is hybridized to the circularized primary probes, and an RCA reaction mixture (containing Phi29 reaction buffer, dNTPs, and Phi29 polymerase) is added to the sample. The sample is incubated at an incubation temperature (e.g., 30° C. or 37° C.) for a defined period of time (e.g. 3 hours), allowing the circularized primary probes to be amplified by the DNA polymerase to generate RCA products, each RCA product comprising multiple copies of a complement of the barcode region of the primary probe. The RCA reaction is terminated by washing the sample with TE buffer.

Sequential hybridization and imaging cycles are performed to identify the locations of target nucleic acid analytes in the sample, and signals are detected and analyzed to reveal the identities of target nucleic acid analytes at the locations. At least one cycle comprises time-gated detection of a detectable label.

In an exemplary cycle with time-gated detection, the biological sample is contacted with a plurality of detectably labeled probes for detecting the multiple analytes, and the detectable labels of the detectably labeled probes are detected. Each detectably labeled probe is configured to hybridize to the complement of the barcode region of a primary probe corresponding to an analyte, and comprises a detectable label. The detectable labels comprise fluorophores, phosphorescent emitters (emitters that emit a phosphorescence signal), and/or thermally activated delayed fluorescence (TADF) emitters, which emit a signal after being stimulated with light. The detectably labeled probes comprise different detectable labels, and two or more of the detectable labels have different signal emission lifetimes, such that signals from the different detectable labels are detectable during different detection time intervals during or after stimulation. In one example, a first detectable label has a first signal emission lifetime that is detectable during a first detection time interval t1 and not detectable during a second detection time interval t2, and a second detectable label has a signal emission lifetime that is detectable during both t1 and t2, for example as shown in FIG. 2E. The detection or absence thereof of a signal at t1 and t2 is used to identify each detectable label at one or more locations, and assign a signal code corresponding to the detectable label at the one or more locations (e.g., as shown in FIG. 1C). Any number of detectable labels can be used, and any two detectable labels can be distinguished based on the detection time interval(s) during which the detectable labels are detected, and/or the channel (e.g., color of detected fluorescent signal) in which the detectable labels are detected. The detection time intervals may be designed in any appropriate configuration, for example as shown in FIGS. 2A-2D.

One or more additional cycles are performed, with signal codes corresponding to the detectable labels assigned to one or more locations in each cycle. After multiple cycles, a sequence of signal codes delineating a signal code sequence is identified at one or more locations in the biological sample. The signal code sequence at a specific location in the biological sample corresponds to the presence of a specific analyte.

An exemplary multi-cycle decoding scheme using time-gated detection of detectable labels is shown in FIG. 4A, as described in detail above. In some aspects, the ability to distinguish detectable labels based on both channel of detection (e.g., signal color) and signal emission lifetime (e.g., using time-gated detection) greatly increases the number of analytes that can be decoded for a given number of cycles.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
a) contacting the biological sample with a plurality of detectably labeled probes for detecting multiple analytes,
wherein each detectably labeled probe is configured to directly or indirectly bind to a different analyte or product thereof and comprises a detectable label, and
wherein the detectable labels of the plurality of detectably labeled probes comprise a first detectable label having a first signal emission lifetime and a second detectable label having a second signal emission lifetime that is longer than the first signal emission lifetime;
b) detecting signals associated with the detectable labels, or absence thereof, during a detection time interval t1 and a detection time interval t2 at one or more locations in the biological sample,
wherein the onset of t2 is later than the onset of t1,
wherein signals associated with the first detectable label are detectable during t1 and not during t2, and
wherein signals associated with the second detectable label are detectable during t2; and
c) generating a signal code sequence comprising signal codes corresponding to the signals or absence thereof detected in step b) during t1 and t2, respectively, at the one or more locations, wherein the signal code sequence corresponds to an analyte of the multiple analytes,
thereby identifying the analyte at the one or more locations in the biological sample.

2. The method of claim 1, wherein the detectable labels of the plurality of detectably labeled probes comprise a third detectable label having a third signal emission lifetime that is longer than the second signal emission lifetime.

3. The method of claim 2, wherein the detecting in step b) further comprises:
detecting signals associated with the detectable labels, or absence thereof, during a detection time interval t3 at one or more locations in the biological sample,
wherein the onset of t3 is later than the onset of t2,
wherein signals associated with the first detectable label and second detectable label are not detectable during t3, and
wherein signals associated with the third detectable label are detectable during t3.

4. The method of claim 3, wherein the signal codes further correspond to the signals or absence thereof detected in step b) during time interval t3 at the one or more locations.

5. A method for analyzing a biological sample, comprising:
a) contacting the biological sample with a plurality of first detectably labeled probes for detecting multiple analytes, wherein each first detectably labeled probe is configured to directly or indirectly bind to a different analyte or product thereof and comprises a detectable label;
b) detecting signals associated with the detectable labels of the plurality of first detectably labeled probes, or absence of the signals, during a detection time interval t1 and a detection time interval t2, at one or more locations in the biological sample,
wherein the detectable labels of the plurality of first detectably labeled probes comprise a first detectable label having a first signal emission lifetime and a second detectable label having a second signal emission lifetime that is longer than the first signal emission lifetime, wherein the onset of t2 is later than the onset of t1, wherein signals associated with the first detectable label are detectable during t1 and not t2, and wherein signals associated with the second detectable label are detectable during t2;
c) contacting the biological sample with a plurality of subsequent detectably labeled probes for detecting the multiple analytes, wherein each subsequent detectably labeled probe is configured to directly or indirectly bind to a different analyte or product thereof and comprises a detectable label; and
d) detecting signals associated with the detectable labels of the plurality of subsequent detectably labeled probes, or absence of the signals, during a subsequent detection time interval t1s and a subsequent detection time interval t2s, at the one or more locations in the biological sample,
wherein the detectable labels of the plurality of subsequent detectably labeled probes comprise a subsequent first detectable label having a subsequent first signal emission lifetime and a subsequent second detectable label having a subsequent second signal emission lifetime that is longer than the subsequent first signal emission lifetime, wherein the onset of t2s is later than the onset of t1s, wherein signals associated with the subsequent first detectable label are detectable during t1s and not t2s, and wherein signals associated with the subsequent second detectable label are detectable during t2s;
wherein a signal code sequence is generated that comprises signal codes corresponding to the signals or absence thereof detected in step b) during t1 and t2, respectively, and step d) during t1s and t2s, respectively, at the one or more locations, wherein the signal code sequence corresponds to an analyte of the multiple analytes,
thereby identifying the analyte at the one or more locations in the biological sample.

6. The method of claim 1, wherein the analyte is a first analyte, the signal code sequence is a first signal code sequence, and wherein a second signal code sequence is generated for a second analyte, the second signal code sequence corresponding to the signals or absence thereof detected at the one or more locations.

7. The method of claim 5, wherein the detection time intervals t1 and t2 comprise the same two time intervals as the subsequent detection time intervals t1s and t2s, respectively.

8.-14. (canceled)

15. The method of claim 1, wherein the detectable signal emitted by each detectable label is fluorescence or phosphorescence.

16.-17. (canceled)

18. The method of claim 1, wherein the signal emission lifetime for each detectable label is a length of time between a) an offset of a stimulus for the detectable label, and b) a time at which the detectable label no longer emits a detectable signal in response to the stimulus.

19. The method of claim 1, wherein the signal emission lifetime of each detectable label is independently selected from: less than about 10 μs, between about 10 μs and about 100 μs, between about 100 μs and about 300 μs, between about 300 μs and about 1 ms, and greater than about 1 ms.

20. (canceled)

21. The method of claim 1, wherein the signals associated with the detectable labels are detected using one or more detection channels, each detection channel being configured to detect light from a different range of wavelengths.

22.-23. (canceled)

24. The method of claim 1, wherein one or more of the detectable labels comprise a thermally activated delayed fluorescence (TADF) emitter and/or a phosphorescent emitter.

25. The method of claim 24, wherein each TADF emitter comprised by a detectable label independently comprises one or more of any of the moieties selected from: DABNA-1; DABNA-2; DTC-DPS; DMAC-DPS; 4CzIPN; NAI-DMAC; DPA-DCPP; an organoboron moiety; a cyanobenzene moiety; a dicyanobenzene moiety; a diphenyltriazine moiety; a diphenylsulfone moiety; a naphthalimide moiety; a dicyanopyrazino moiety; a phenanthrene moiety; a carbazole moiety optionally substituted with C₁-C₆ alkyl; a phenoxazine moiety optionally substituted with C₁-C₆ alkyl; a triphenylamine moiety optionally substituted with C₁-C₆ alkyl; a diphenylamide moiety optionally substituted with C₁-C₆ alkyl; an acridine moiety optionally substituted with C₁-C₆ alkyl; and a dimethylacridine moiety optionally substituted with C₁-C₆ alkyl.

26. The method of claim 24, wherein one or more of the TADF emitters comprised by a detectable label is encapsulated in a polymer matrix.

27.-31. (canceled)

32. The method of claim 1, wherein each analyte is independently a nucleic acid analyte.

33. The method of claim 1, wherein each detectably labeled probe (a) binds to a primary probe that directly binds to its corresponding analyte, or (b) binds to an intermediate probe that binds directly or indirectly to a primary probe that directly binds to its corresponding analyte.

34. The method of claim 33, wherein the primary probe and the intermediate probe are independently selected from the group consisting of: a probe comprising a 3′ or 5′ overhang, wherein the 3′ or 5′ overhang comprises one or more barcode sequences; a probe comprising a 3′ overhang and a 5′ overhang, wherein the 3′ overhang and the 5′ overhang each independently comprises one or more barcode sequences; a circular probe; a circularizable probe or probe set; a probe or probe set comprising a split hybridization region configured to hybridize to a splint; and a combination thereof.

35. The method of claim 1, wherein the product of each analyte is a rolling circle amplification (RCA) product generated in situ in the biological sample.

36. The method of claim 1, wherein the biological sample is non homogenized and optionally selected from the group consisting of a formalin-fixed, paraffin-embedded (FFPE) sample, a frozen tissue sample, or a fresh tissue sample.

37.-42. (canceled)

43. The method of claim 1, wherein the method is performed in situ in the biological sample.

44.-50. (canceled)
